(19) United States
(12) Patent Application Publication
(10) Pub. No.: US 2026/0144644 A1
(43) Pub. Date: May 28, 2026

(54) PIEZOELECTRIC SACROILIAC JOINT FUSION IMPLANT ASSEMBLY AND KIT FOR MAKING SAME

(71) Applicant: Spark Assets LLC, Needham, MA (US)

(72) Inventors: Ziev Moses, Needham, MA (US); Kevin Chappuis, Saugus, MA (US); Luke Diehl, Medford, MA (US)

(73) Assignee: Spark Assets LLC, Needham, MA (US)

(21) Appl. No.: 19/398,313

(22) Filed: Nov. 24, 2025

Related U.S. Application Data
(60) Provisional application No. 63/723,713, filed on Nov. 22, 2024.

(51) Int. Cl. A61F 2/44; A61F 2/30; A61L 27/06; A61L 27/16; A61L 27/34; B33Y 80/00

(52) U.S. Cl. CPC A61F 2/4455; A61F 2/30771; A61F 2/3094; A61L 27/06; A61L 27/16; B33Y 80/00; A61F 2002/30087; A61F 2002/30156; A61F 2002/30383; A61F 2002/30433; A61F 2002/30617; A61F 2002/3092; A61F 2002/3093; A61F 2002/30985; A61L 2400/18

(57) ABSTRACT

A sacroiliac (SI) joint implant assembly addresses SI joint dysfunction and degenerative sacroiliitis. The assembly includes a first implant body, a second implant body, and a piezoelectric layer positioned between them. The piezoelectric layer generates electrical charges when subjected to mechanical stress, stimulating bone growth and enhancing stability of the SI joint after surgery. The implant bodies may feature non-planar surfaces to distribute compressive and shear forces unevenly across the piezoelectric layer. The assembly can be secured using fasteners such as screws, pins, or brackets, and may include porous or fenestrated bodies made from materials like titanium or polymers. Some implant assemblies are designed for minimally invasive surgical procedures, with configurations that allow for precise placement using guide wires and imaging techniques. The assembly aims to stabilize the SI joint and promote bone fusion, leveraging the piezoelectric effect to improve the overall efficacy of the fusion procedure.

### CROSS-REFERENCE TO RELATED APPLICATION

This application claims the benefit of priority under 35 U.S.C. § 119(e) of U.S. Provisional Application Ser. No. 63/723,713, filed Nov. 22, 2024, entitled “PIEZOELECTRIC SACROILIAC JOIN FUSION IMPLANT ASSEMBLY AND KIT FOR MAKING SAME.” The disclosure of the prior application is considered a part of and is incorporated by reference in its entirety in the disclosure of this application.

### FIELD

The present application relates to medical devices, for example to sacroiliac (SI) joint implants to address SI joint dysfunction and degenerative sacroiliitis.

### BACKGROUND

Conditions such as sacroiliac (SI) joint dysfunction arise from SI joint disruption and degenerative sacroiliitis. The SI joint, located in the pelvis, connects the ilium to the sacrum, the part of the spine above the tailbone. This joint plays a role in transferring weight and forces between the upper body and legs, contributing to shock absorption and energy transfer during activities like walking.

Approximately 20 to 25% of patients with low back complaints have issues originating from the SI joint. Studies indicate that the incidence of SI joint degeneration after lumbar fusion surgery reaches 75% at five years post-surgery. When non-surgical therapies such as physical therapy, oral medications, or injection therapy fail, minimally invasive procedures become a viable option.

The state of the art provides mainly two types of SI joint implants: dowels and screws, with a preference for screw-based systems. The implants aim to fuse the SI joint by providing stability thereto.

### BRIEF DESCRIPTION OF THE FIGURES

Further embodiments of the present application can be understood with reference to the appended figures.

FIG. 1 shows a pelvis after a sacroiliac joint with SI joint implants inserted to stabilize the joint.

FIG. 2 shows SI dowel-shaped joint implants according to the state of the art.

FIG. 3 shows an environment for an SI joint fusion surgical procedure.

FIG. 4A shows an exploded perspective view of a kit 400A to form an SI joint implant assembly according to a first embodiment.

FIG. 4B shows a perspective view of an SI joint implant assembly 400B formed from assembling the kit 400A according to the first embodiment, being guided over a guide wire 315.

FIG. 5 is an exploded perspective view of a kit 500 to form an SI joint implant assembly according to a second embodiment.

FIG. 6 shows a perspective view of an SI joint implant assembly 600 formed from assembling the kit according to the first embodiment.

FIG. 7 shows a perspective view of an SI joint implant assembly 700 according to a third embodiment.

FIG. 8 shows a perspective view of an SI joint implant assembly 800 according to a fourth embodiment.

FIG. 9A is an exploded perspective view of a kit 900A to form an SI joint implant assembly according to a fifth embodiment.

FIG. 9B is another exploded perspective view of kit 900A to form an SI joint implant assembly according to the fifth embodiment.

FIG. 9C shows a side cross-sectional view of an SI joint implant assembly 900C formed from assembling a variation of kit 900A.

FIG. 10A is an exploded perspective view of a kit 1000A to form an SI joint implant assembly according to a sixth embodiment.

FIG. 10B shows a perspective view of an SI joint implant assembly 1000B formed from assembling the kit 1000A according to the sixth embodiment.

FIG. 11 is an exploded perspective view of a kit 1100A to form an SI joint implant assembly according to a seventh embodiment.

FIG. 12 shows an exploded perspective view of a kit 1200 to form an SI joint implant assembly according to an eighth embodiment.

FIG. 13 shows an exploded perspective view of a kit 1300 to form an SI joint implant assembly according to a ninth embodiment.

FIG. 14A shows an environment 1400A for an SI joint fusion surgical procedure using SI implant assemblies 800 of FIG. 8.

FIG. 14B shows a perspective view of SI joint implant assembly 800 of FIG. 8 with a double charge along the “target zone” positioned at the joint.

FIG. 15A shows an exploded perspective view of a kit 1500A to form an SI joint implant assembly according to a tenth embodiment.

FIG. 15B shows a perspective view of an SI joint implant assembly 1500B formed from assembling the kit 1500A according to the tenth embodiment.

FIG. 16A is an exploded perspective view of a kit 1600A to form an SI joint implant assembly according to a eleventh embodiment.

FIG. 16B shows a perspective view of an SI joint implant assembly 1600B formed from assembling the kit 1600A according to the eleventh embodiment.

FIG. 17A shows an exploded perspective view of a kit 1700A to form an SI joint implant assembly according to an twelfth embodiment.

FIG. 17B shows a perspective view of an SI joint implant assembly 1700B formed from assembling the kit 1700A according to the twelfth embodiment.

FIG. 18 shows an exploded perspective view of a kit 1800 to form an SI joint implant assembly according to a thirteenth embodiment.

FIG. 19 is an exploded perspective view of a kit 1900 to form an SI joint implant assembly according to a fourteenth embodiment.

FIG. 20A shows a perspective view of an SI joint implant assembly 2000 formed from assembling the kit according to the fifteenth embodiment.

FIG. 20B shows a perspective view of an SI joint implant assembly 2000 formed from assembling the kit according to the fifteenth embodiment.

FIG. 21A shows a perspective view of a screw assembly 2100A for use in an orthopedic implant assembly.

FIG. 21B shows an exploded perspective view of a kit 2100B to form a screw assembly 2100A for use in an orthopedic implant assembly.

FIG. 21C shows an exploded perspective view of a screw assembly 2100B for use in an orthopedic implant assembly.

### DETAILED DESCRIPTION

A sacroiliac (SI) joint fusion implant assembly is designed to address SI joint dysfunction and degenerative sacroiliitis. The SI joint, located in the pelvis, is crucial for weight transfer and shock absorption. The invention includes various configurations of SI joint implants, which may feature non-planar surfaces to distribute compressive and shear forces unevenly across a piezoelectric layer. The implants can be assembled using fasteners like screws, pins, or brackets, and may include porous or fenestrated bodies made from materials such as titanium or polymers. The document details different embodiments of the implant, including configurations with multiple piezoelectric layers and various shapes and cross-sections for the implant bodies and fasteners. The implants aim to stabilize the SI joint and promote bone fusion, with the potential for minimally invasive surgical procedures.

Some embodiments address the design and functionality of a sacroiliac (SI) joint fusion assembly, focusing on the use of piezoelectric materials to enhance bone growth and stability. The assembly includes a first implant body (e.g., a proximal implant body or an inner implant body), a second implant body (e.g., a distal implant body or an outer implant body), and with one or multiple piezoelectric layers (which could be in the form of a flat layer, a curved layer including a sleeve layer), which generate electrical charges to stimulate bone growth. The implant assemblies are designed to be inserted between the ilium and sacrum using a guide wire to extend into a cannula of the SI joint implant assembly. According to some embodiments, the cannula could be provided inside a fastener of the SI joint implant assembly, such as a screw, or inside an implant body of the SI joint implant assembly.

According to some embodiments, the first implant body and/or the second implant body may be porous.

According to some embodiments, the first implant body and/or the second implant body may include an electrically conductive material, such as, for example, titanium.

According to some embodiments, the first implant body and/or the second implant body may be 3D printed.

According to some embodiments, one or more of the first and second implant bodies and or the fastener may include a fenestration/be fenestrated.

According to some embodiments, fenestration openings between the first and second implant bodies may be in registration with one another in the SI joint implant assembly to promote bone growth through the SI joint implant assembly.

According to some embodiments, one or more of the first implant body, the second implant body and the fastener may including a threaded portion.

According to some embodiments, the fastener may include one or more fasteners.

According to some embodiments, where the fastener includes one or more screws, the one or more screws may exhibit a screw having threads as being uniform down the entire length of the one or more screws, or variable in pitch or thread count down the length of the one or more screws to optimize use in different bone densities. For example, in hard bone the device would provide further mechanical stability with tightly spaced threads or threads with a fine pitch. Alternatively, in soft bone the device would get superior purchase if the threads were further spaced out. If the device is to anchor into anatomy with variable bone density it would be beneficial to the device performance to have tighter threads where the bone is more dense and courser threads where the bone is less dense.

According to some embodiments, the one or more piezoelectric layers of the SI joint implant assembly may be flat before assembly (in the “kit” of the SI joint implant assembly), and may then assume a non-planar shape by conforming to the non-planar shape of the implant body surfaces adjacent thereto in the assembly.

According to some other embodiments, the one or more piezoelectric layers of the SI joint implant assembly kit may have surfaces that match the surface configurations of the implant body surfaces adjacent thereto in the assembly. This, according to such embodiments, where the implant body adjacent surfaces are non-planar, the one or more piezoelectric layers may be non-planar in the kit as well, prior to assembly.

Some embodiments contemplate the use of imaging markers on the implants such that, during surgery, the implant assembly can be accurately placed.

Some embodiments maintain electrical isolation between different parts (implant bodies/fastener/piezoelectric layer) of the implant assembly to ensure effective charge distribution.

Any one of the piezoelectric layers as described herein may be substantially planar in shape prior to assembly, and deformed into a non-planar shape, such as having undulations, waves, zig-zags, curves, creases, and/or bends, due to the application of a force to the proximal and distal SI joint implant bodies. The piezoelectric components may conform to the inner surfaces of the SI joint implant bodies.

Proximal and distal SI joint implant bodies, fasteners such as screws, pins and/or brackets as described herein may be unitary (i.e., a one piece component) or they may have multiple attached components that together form such components. Such components may be formed, for example, using 3D printing, 3D machining, or any other suitable technique.

A screw as disclosed herein could have any configuration, such as a lag screws or any other type of screw based on application needs. Screws could include an electrically conductive material, such as titanium, or they could include an electrically insulating material, such as PEEK, or another polymeric material.

Although embodiments as shown depict an SI joint implant assembly and/or SI implant bodies that are either substantially triangular or substantially circular in a plan transverse to a longitudinal direction thereof, embodiments are not so limited, and include within the their scope an SI joint implant assembly and/or SI implant bodies having such cross-section that is not triangular, such as a polygonal, square, star-shaped cross-section, or such as a cross section that changes shape along a longitudinal direction of the SI joint implant assembly.

In some of the shown embodiments, although pins are depicted as having a cylindrical cross section, embodiments are not so limited, and include within their scope the provision of pins of any cross section, such as oval, polygonal, rectangular, square, star-shaped, etc.

As used herein, a “kit” refers to the components of an SI joint implant assembly prior to final assembly of such components into the SI joint implant assembly.

As used herein, “electrically conductive” in some examples may refer to a property of a material having an electrical conductivity greater than or equal to 106 Siemens per meter (S/m) at 20 degrees Celsius. Examples of such materials include titanium (with relatively low electrical conductivity compared to many other metals, that is, about 2.4×106 S/m) and tungsten (with much higher electrical conductivity of about 1.8×107 S/m).

As used herein, a “material” may include one or more different materials.

As used herein, an “electrically conductive material” may include one or more of titanium, tungsten, stainless steel, platinum or gold, it being understood that the latter list of materials is to include alloys of the same.

As used herein, an “electrically insulating material” may include a polymer, such as, for example, PEEK (Polyether Ether Ketone), Polyimide (PI), Polyphenylene Sulfide (PPS), Polyaryletherketone (PAEK), Polyetherketone (PEK), Polyetherketoneketone (PEKK), and/or Fluoropolymers.

As used herein, holes (or lumens) that “register” or that are in “registration” with one another are holes that are either in full registration (holes that overlap one another fully) with one another, or in partial registration with one another (holes that overlap one another partially).

Holes or lumens as described herein may be through holes or blind holes. Such holes may be provided, for example, by way of machining, or using any other suitable technique.

An “irregular surface” or a “surface with irregularities” of an implant body as used herein may refer to a surface that is ribbed, ridged, grooved, embossed, undulated, or striated, in such a way that it may mechanically engage mating/conforming irregularities in an adjacent surface.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

By “non-planar” in the context of a surface, what is meant herein is a surface that is not flat, such as a surface with at least one of undulations/waves, zig-zags, curves, bends, corners, or creases, oriented in any direction. For example, a non-planar surface may be concave or convex. The surfaces may be smooth, or they may contain any number of sharp features.

In the context of embodiments, a non-planar interface with a piezoelectric layer is to distribute compressive or shear forces applied to the implant bodies unevenly across the piezoelectric layer.

In order to arrive at embodiment, through experimentation, inventors have discovered that, a same axial/compressive force, a same shear force, and a same force combining the two, when applied to two implant bodies of an SI joint implant assembly with a piezoelectric layer therebetween, result in the generation of a higher voltage output across the piezoelectric layer when the piezoelectric layer defines, in the assembly, 2D (2-dimensional) longitudinally extending undulations/wave patterns as opposed to planar inner surfaces or to inner surfaces with a single wave. In addition, inventors have discovered that, a same axial/compressive force, a same shear force, and a same force combining the two, when applied to implant bodies of an SI joint implant assembly with a piezoelectric layer therebetween, result in the generation of a higher voltage output across the piezoelectric layer when the piezoelectric layer defines, at respective inner surfaces thereof, 3D (3 -dimensional) undulations/wave patterns (i.e., extending both longitudinally and both transversely to the longitudinal direction and horizontally) as opposed to planar inner surfaces or to inner surfaces with a single wave. Inventors has discovered that such increased voltage outputs advantageously result in associated improved (e.g., faster and more copious) osteogenesis within the body.

Advantageously, a non-planar configuration of the piezoelectric layer when it is assembled into an SI joint implant assembly according to some embodiments ultimately results in improved osteogenesis as compared with spinal implant assemblies of the prior art where the piezoelectric layer is flat.

When non-surgical therapies such as physical therapy, oral medications, or injection therapy fail, minimally invasive SI joint fusion becomes a viable option. Existing implant configurations are designed to stabilize and fuse the heavily loaded SI joint. However, these current solutions have several shortcomings. They often fail to provide adequate long-term stability and may not effectively promote bone growth across the joint.

The disclosed SI joint implant assembly according to some embodiments addresses these issues by providing novel configurations that enhance stability and promote bone growth.

According to some embodiments, the assembly includes proximal and distal SI joint implant bodies, an intermediate implant body, and one or more piezoelectric layers. The surfaces of the implant bodies adjacent to the piezoelectric layers may be non-planar, ensuring uneven distribution of compressive and shear forces across the piezoelectric layers. This configuration not only stabilizes the SI joint but also leverages the piezoelectric effect to stimulate bone growth, thereby improving the overall efficacy of the fusion procedure.

Piezoelectric materials generate electrical charges when subjected to mechanical stress, which can stimulate bone growth. The implants can be assembled using fasteners like screws, pins, or brackets, and may include porous or fenestrated bodies made from materials such as titanium or polymers. The porous nature of SI implant assemblies can facilitate bone growth through the SI joint implant assembly, promoting fusion between the sacrum and ilium.

Some embodiments address the design and functionality of an SI joint fusion assembly, focusing on the use of piezoelectric materials to enhance bone growth and stability. Embodiments described herein may include one or multiple piezoelectric layers, which generate electrical charges to stimulate bone growth.

Before explaining at least one aspect of the disclosed and/or claimed inventive concept(s) in detail, it is to be understood that the disclosed and/or claimed inventive concept(s) is not limited in its application to the details of construction and the arrangement of the components or steps or methodologies set forth in the following description or illustrated in the drawings. The disclosed and/or claimed inventive concept(s) is capable of other aspects or of being practiced or carried out in various ways. Various embodiments containing different features and aspects are shown in this disclosure. It should be understood that other embodiments containing some of these features and aspects, and other features and aspects known in the art, are within the scope of this disclosure, even if such an exact combination is not shown or described specifically. Also, it is to be understood that the phraseology and terminology employed herein is for the purpose of description and should not be regarded as limiting.

As utilized in accordance with the disclosure, the following terms, unless otherwise indicated, shall be understood to have the following meanings. Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular. The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out embodiments described herein. All percentages, parts, proportions, and ratios as used herein, are by weight of the total composition, unless otherwise specified. All such weights as they pertain to listed ingredients are based on the active level and, therefore; do not include solvents or by-products that may be included in commercially available materials, unless otherwise specified.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or value beyond those recited. Headings, lists, and numbering included herein are for ease of explanation only and are not meant to be limiting.

It will also be understood that, although the terms “first,” “second,” etc. may be used herein to describe various elements, these elements should not be limited by these terms. These terms are only used to distinguish one element from another. For example, a first node could be termed a second node, and, similarly, a second node could be termed a first node, which changing the meaning of the description, so long as all occurrences of the “first node” are renamed consistently and all occurrences of the “second node” are renamed consistently. The first node and the second node are both nodes, but they are not the same node.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B, and C). In embodiments, the phrase “A is located on B” means that at least a part of A is in direct physical contact or indirect physical contact (having one or more other features between A and B) with at least a part of B. In the instant description, “A is adjacent to B” means that at least part of A is in direct physical contact with at least a part of B. In the instant description, “B is between A and C” means that at least part of B is in or along a space separating A and C and that the at least part of B is in direct or indirect physical contact with A and C. In the instant description, “A is attached to B” means that at least part of A is mechanically attached to at least part of B, either directly or indirectly (having one or more other features between A and B). In the instant description, “the As are coupled to the Bs” means that at least some of the As are coupled to at least some of the Bs, and not necessarily that all As are coupled to at least one B and all Bs are coupled to at least one A. In the instant description, “A is within B” means that at least some of A is encompassed within the physical boundaries of B. The use of reference numerals separated by a “/”, such as “102/104” for example, is intended to refer to 102 or 104 as appropriate. Otherwise, the forward slash (“/”) as used herein means “and/or.”

When used to describe a range of dimensions, the phrase “between X and Y” represents a range that includes X and Y. Although certain elements may be referred to in the singular herein, such elements may include multiple sub-elements. For example, “an insulating material” may include one or more insulating materials.

As used herein, “an insulating material” refers to an electrically insulating material.

As used herein, “a conductive material” refers to an electrically conductive material.

The use of the techniques and structures provided herein can be detected using tools such as: electron microscopy including scanning/transmission electron microscopy (SEM/TEM), scanning transmission electron microscopy (STEM), nano-beam electron diffraction (NBD or NBED), and reflection electron microscopy (REM); composition mapping; x-ray crystallography or diffraction (XRD); energy-dispersive x-ray spectroscopy (EDX); secondary ion mass spectrometry (SIMS); time-of-flight SIMS (ToF-SIMS); atom probe imaging or tomography; local electrode atom probe (LEAP) techniques; 3D tomography; or high resolution physical or chemical analysis, to name a few suitable example analytical tools. In particular, such tools can indicate an integrated circuit including at least one semiconductor package including an embedded magnetic inductor. In some embodiments, the techniques, processes and/or methods described herein can be detected based on the structures formed therefrom. In addition, in some embodiments, the techniques and structures described herein can be detected based on the benefits derived therefrom. Numerous configurations and variations will be apparent in light of this disclosure.

The description may use the phrases “in an embodiment,” “according to some embodiments,” “in accordance with embodiments,” or “in embodiments,” which may each refer to one or more of the same or different embodiments. Furthermore, the terms “comprising,” “including,” “having,” and the like, as used with respect to embodiments of the present disclosure, are synonymous. The foregoing description and summary are to be understood as being in every respect illustrative and exemplary, but not restrictive, and the scope disclosed herein is not to be determined only from the detailed description of illustrative implementations but according to the full breadth permitted by patent laws. It is to be understood that the implementations shown and described herein are only illustrative of the principles of the present invention and that various modifications may be implemented by those skilled in the art without departing from the scope and spirit.

By a non-planar surface A “conforming to” another non-planar surface B, what is meant herein is that surface A and surface B exhibit inter-fitting surfaces, enabling a complementary nesting configuration of surfaces A and B. In the context of embodiments, one or more non-planar surfaces of an SI joint implant body that is used to make an SI joint implant assembly may be configured such that, when subjected to compressive and/or shear forces, they apply compressive and/or shear forces to a piezoelectric layer adjacent thereto in an unevenly distributed manner across the piezoelectric layer.

A fastener may include any structure to fasten two implant bodies together, such as screws, rivets, pins, teeth to be mated, brackets, etc. In some of the shown embodiments, although holes within proximal and/or distal SI joint implant bodies are sometimes depicted as having a cylindrical cross section, embodiments are not so limited, and include within their scope the provision of holes of any cross section, such as oval, polygonal, rectangular, square, star-shaped, etc. In some of the shown embodiments, although pins are depicted as having a cylindrical cross section, embodiments are not so limited, and include within their scope the provision of pins of any cross section, such as oval, polygonal, rectangular, square, star-shaped, etc.

Embodiments include within their scope any of the SI joint implant body configurations and/or fastener solutions as shown as described herein wherein the SI joint implant assembly includes proximal and distal SI joint implant bodies, plus, optionally, one or more intermediate SI joint implant bodies distinct from a piezoelectric layer in between the proximal and distal SI joint implant bodies. According to one embodiment, surfaces of the SI joint implant that are adjacent to surfaces of the piezoelectric layer may be non-planar. In the context of embodiments, a non-planar interface with a piezoelectric layer is to distribute compressive and/or shear forces applied to the implant unevenly across the piezoelectric layer. In the context of the instant description, the words “interface” or “seam” or “boundary” between A and B do not necessarily mean that A and B must be directly physically touching/adjacent, although they could. Rather, these words pertain to an interaction between surfaces of A and B, whether direct or indirect.

Any implant body could be porous, non-porous, fenestrated, not fenestrated, and/or include titanium, or another suitable electrically conductive material.

Porous as used herein refers to a material defining small openings or pores, typically ranging from 150 to 900 microns in diameter, with an optimal pore size around 500 microns ±200 microns.

Fenestrated as used herein refers to an object that defines one or more recesses or openings in walls thereof.

Components of a SI joint assembly according to embodiments may include one or more biocompatible materials.

A piezoelectric layer as referred to herein may include one or more piezoelectric materials, such as, for example, a polymer exhibiting piezoelectric characteristics. For example, a piezoelectric material as referred to herein may include a piezoelectric polymer, such as, by way of example, polyvinylidene difluoride (PVDF). A piezoelectric layer as referred to herein may include one or more layers, may include one or more non-piezoelectric materials in addition to at least one piezoelectric material, and it may have a uniform or non-uniform material composition throughout.

In the figures, like components are marked with the same or with similar reference numerals. As a result, when a certain component has already been described with respect to one figure, it may not be expressly described again with respect to another figure that includes the same certain component.

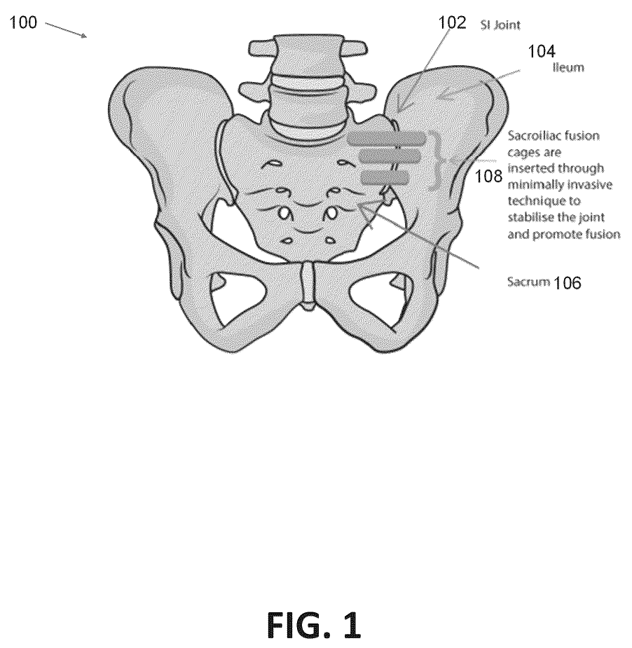

FIG. 1 shows a pelvis 100 after a sacroiliac joint procedure. The sacroiliac (SI) joint 102 is located between the ilium 104 and the sacrum 106. The SI joint 102 is a structure that transfers weight and forces between the upper body and the legs, playing a significant role in shock absorption and energy transfer during activities such as walking.

The figure illustrates the placement of SI joint implants or SI fusion cages 108. These implants are inserted across the SI joint 102 through a minimally invasive technique. The primary purpose of these implants is to stabilize the joint and promote fusion between the ilium 104 and the sacrum 106. This procedure is typically considered when non-surgical therapies, such as physical therapy, oral medications, or injection therapy, fail to provide relief from SI joint pain and instability.

The SI joint implants 108 are designed to stop abnormal movement between the sacrum 106 and the ilium 104, thereby alleviating pain and improving joint stability. The minimally invasive nature of the procedure allows for a smaller incision, reduced recovery time, and less postoperative discomfort compared to traditional open surgical methods.

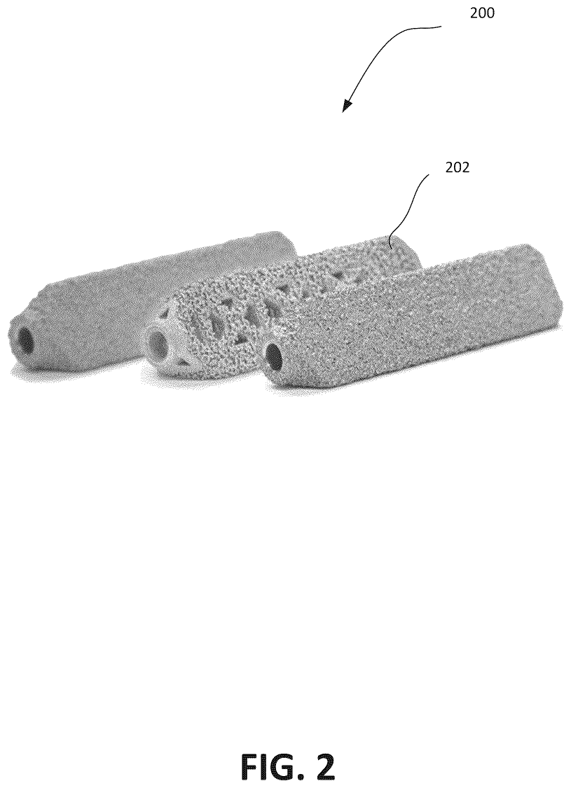

FIG. 2 shows some SI joint implants 200 according to the state of the art, which are designed for sacroiliac joint fusion procedures. These implants are dowel-shaped and are constructed from a porous titanium material.

The middle SI joint implant 202 features fenestrated walls, which provide additional pathways for bone growth. The fenestrations enhance the integration of the implant with the surrounding bone, further stabilizing the joint. The cannulated design of the implants includes a longitudinally extending lumen, which allows for the insertion of a guide wire during the surgical procedure. This guide wire aids in the precise placement of the implants across the SI joint.

The rigid titanium construction and the triangular cross-section of the implants provide structural stability to the SI joint. The geometry of the implants ensures that they remain securely in place, preventing rotational movement and maintaining the alignment of the sacrum and ilium. The use of these implants in minimally invasive SI joint fusion procedures aims to alleviate pain and improve joint stability by promoting bone fusion and reducing abnormal joint movement.

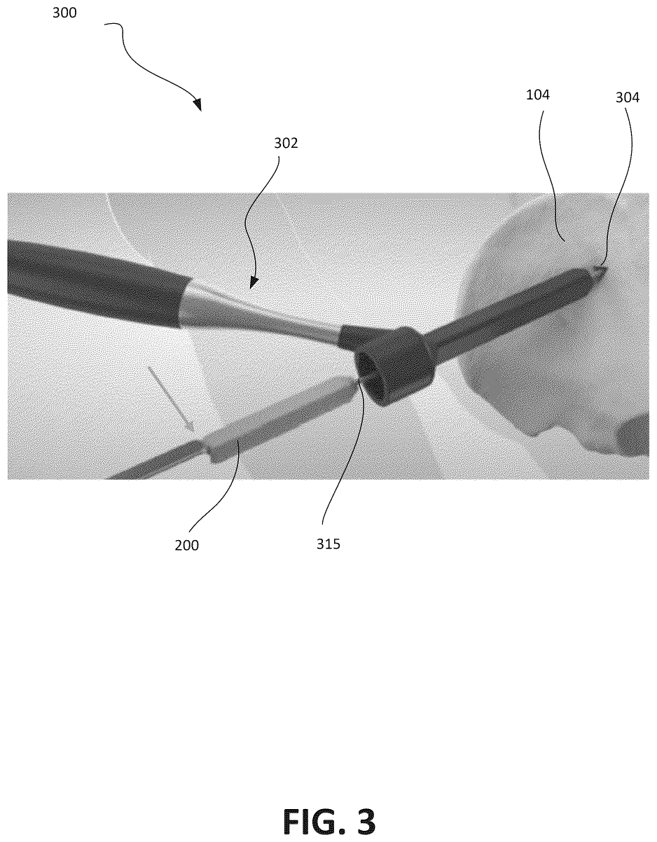

FIG. 3 shows an environment 300 for an SI joint fusion surgical procedure. The procedure is performed in an operating room with either general or SI joint anesthesia. The patient lies face down during the surgery while the surgeon uses a specially designed system 302 to guide the instruments that prepare the bone and insert the implant 200.

The system 302 includes a guide wire 315 that extends through the lumen of each SI joint implant to guide the same to the target implantation site through an opening 304 made in the sacrum 102. The entire procedure is performed through a small 2 to 3 centimeter incision along the side of the buttocks. During the procedure, fluoroscopy can provide the surgeon with live imaging to facilitate proper placement of the implant 200. Fluoroscopy is an imaging technique commonly used by physicians to obtain real-time images of internal structures using X-rays. Normally, three implants will be used depending on the patient's size and anatomy.

For the procedure, a chisel implement may be used to create a hole in the sacrum, the hole to correspond for example to a cross-sectional shape of the implant. Then, the implant may be hammer into the hole to extend to the SI joint. The bones of the ilium and sacrum at the SI join region are porous, and would admit such implantation.

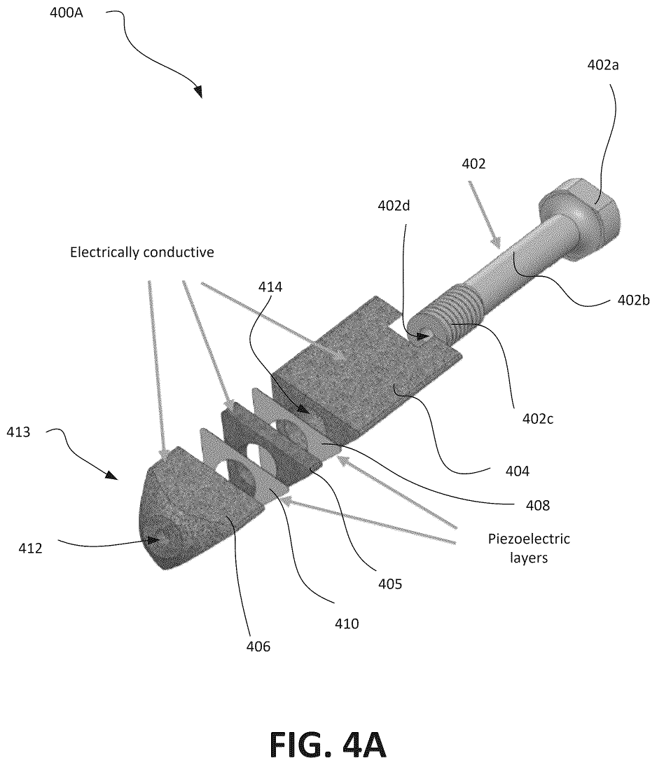

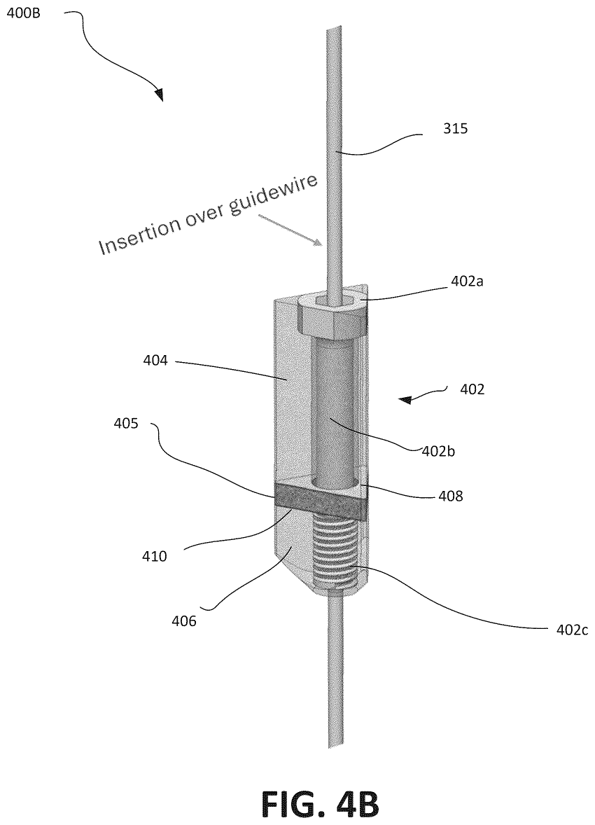

FIG. 4A shows an exploded perspective view of a kit 400A to form an SI joint implant assembly according to a first embodiment. The kit 400A includes a lag screw 402, which comprises a screw head 402a, a smooth screw shaft portion 402b, a threaded screw shaft portion 402c, and a screw lumen 402d. The screw lumen 402d is cannulated to accommodate a guide wire, facilitating precise placement during the surgical procedure.

The kit 400A further includes a proximal implant body 404, an intermediate implant body 405, and a distal implant body 406. Each of these implant bodies includes an electrically conductive material. The proximal implant body 404 features a longitudinal lumen 414 with a smooth inner surface, designed to receive the smooth screw shaft portion 402b. The distal implant body 406 includes a longitudinal lumen 412 with threaded inner walls to engage the threaded screw shaft portion 402c. The diameter of lumen 414 is to be larger than that of the smooth shaft portion 402b in order to prevent a short between the implant bodies.

Between the proximal implant body 404 and the intermediate implant body 405, as well as between the intermediate implant body 405 and the distal implant body 406, are first and second piezoelectric layers 408 and 410, respectively. These piezoelectric layers generate electrical charges when subjected to mechanical stress, which can stimulate bone growth and enhance the stability of the SI joint fusion. The piezoelectric layers 408 and 410 are designed to conform to the adjacent surfaces of the implant bodies, ensuring effective force distribution and electrical charge generation.

The assembly of the kit 400A involves aligning the lumens 402d, 412, and 414, along with the lumens or holes provided in the piezoelectric layers 408 and 410, to form a continuous passage for the guide wire. This alignment ensures that the implant bodies and piezoelectric layers are correctly positioned relative to each other, facilitating the insertion and stabilization of the implant assembly within the SI joint.

FIG. 4B shows a perspective view of an SI joint implant assembly 400B formed from assembling the kit 400A according to the first embodiment. The assembly 400B is being guided over a guide wire 315, which extends through the entire length of the implant assembly, facilitating precise placement during the surgical procedure. The guide wire 315 facilitates the insertion and stabilization of the implant assembly within the SI joint.

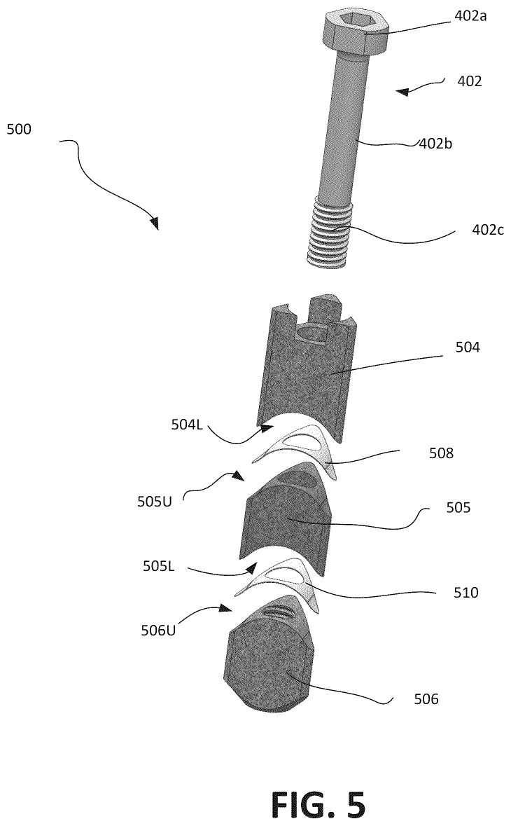

FIG. 5 shows an exploded perspective view of a kit 500 to form an SI joint implant assembly according to a second embodiment.

The kit 500 includes a proximal implant body 504, an intermediate implant body 505, and a distal implant body 506. Each of these implant bodies includes an electrically conductive material. The proximal implant body 504 features a lower surface 504L, which is concave. The intermediate implant body 505 has an upper surface 505U that is convex and conforms to the lower surface 504L of the proximal implant body 504. The lower surface 505L of the intermediate implant body 505 is also concave, while the upper surface 506U of the distal implant body 506 is convex and conforms to the lower surface 505L of the intermediate implant body 505.

Between the proximal implant body 504 and the intermediate implant body 505, as well as between the intermediate implant body 505 and the distal implant body 506, are first and second piezoelectric layers 508 and 510, respectively. These piezoelectric layers generate electrical charges when subjected to mechanical stress, which can stimulate bone growth and enhance the stability of the SI joint fusion. The non-planar surfaces of the implant bodies ensure uneven distribution of compressive and shear forces across the piezoelectric layers, enhancing the overall stability and promoting bone growth. The piezoelectric layers 508 and 510 are designed to conform to the adjacent surfaces of the implant bodies, ensuring effective force distribution and electrical charge generation.

The assembly of the kit 500 involves aligning the implant bodies and piezoelectric layers to form a continuous structure that can be inserted into the SI joint. The lag screw 402 secures the implant bodies together, maintaining the alignment and structural integrity of the assembly.

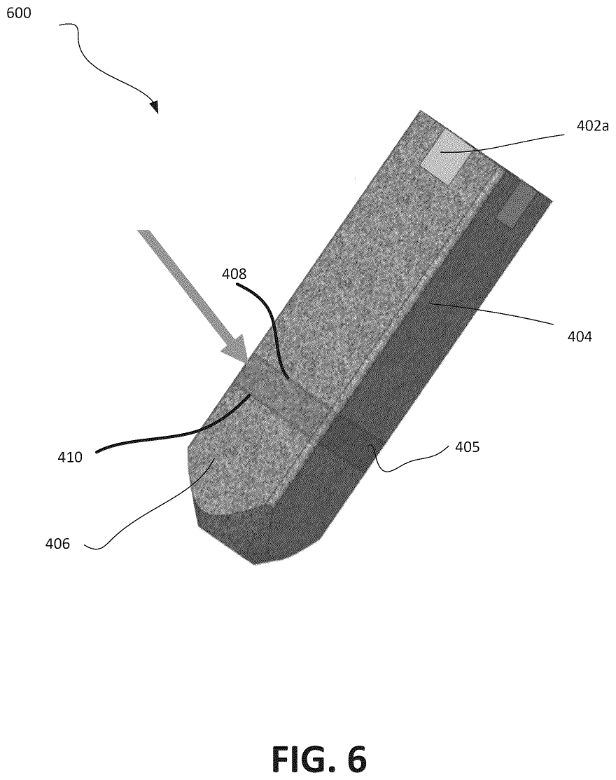

FIG. 6 shows an SI joint implant assembly 600 formed from assembling the kit according to the first embodiment. In this embodiment, even though the surfaces interfacing with the piezoelectric layers are planar, the presence of the piezoelectric layers advantageously promotes bone growth by virtue of electrical charge generation.

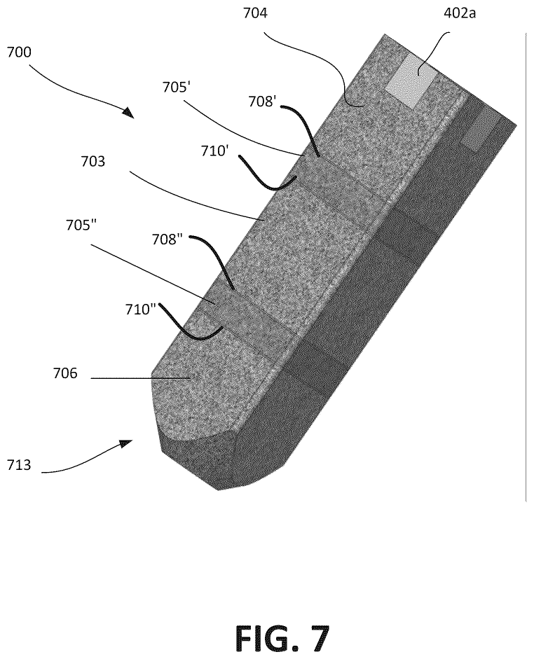

FIG. 7 shows an SI joint implant assembly 700 according to a third embodiment. The assembly 700 includes a proximal implant body 704, a first intermediate implant body 705′, a middle implant body 703, a second intermediate implant body 705″, and a distal implant body 706. The assembly also features a first proximal piezoelectric layer 708′, a second proximal piezoelectric layer 710′, a first distal piezoelectric layer 708″, and a second distal piezoelectric layer 710″.

The proximal implant body 704, first intermediate implant body 705′, middle implant body 703, second intermediate implant body 705″, and distal implant body 706 include an electrically conductive material. The piezoelectric layers 708′, 710′, 708″, and 710″ are positioned between these implant bodies to generate electrical charges when subjected to mechanical stress, thereby promoting bone growth and enhancing the stability of the SI joint fusion.

The assembly 700 is designed to be inserted into the SI joint, with the tapered nose 713 facilitating the insertion process. The piezoelectric layers conform to the adjacent surfaces of the implant bodies, ensuring effective force distribution and electrical charge generation. Even though the surfaces interfacing with the piezoelectric layers are planar, the presence of the piezoelectric layers advantageously promotes bone growth by virtue of electrical charge generation.

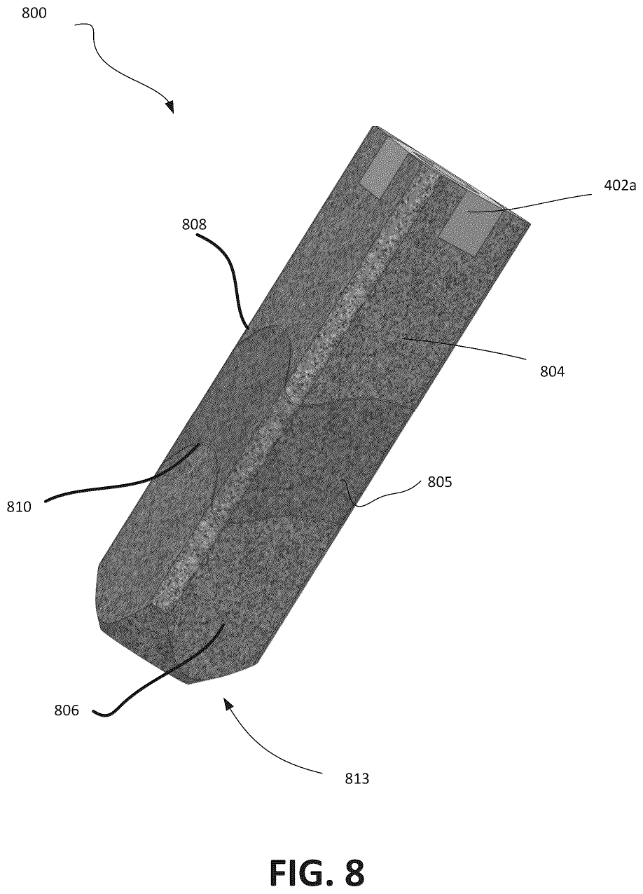

FIG. 8 shows an SI joint implant assembly 800 according to a fourth embodiment, which includes a proximal implant body 804, an intermediate implant body 805, a distal implant body 806, a first piezoelectric layer 808, a second piezoelectric layer 810, and a truncated nose 813.

The proximal implant body 804, intermediate implant body 805, and distal implant body 806 include an electrically conductive material. The first piezoelectric layer 808 is positioned between the proximal implant body 804 and the intermediate implant body 805, while the second piezoelectric layer 810 is positioned between the intermediate implant body 805 and the distal implant body 806. These piezoelectric layers generate electrical charges when subjected to mechanical stress, which can stimulate bone growth and enhance the stability of the SI joint fusion.

The surfaces of the proximal implant body 804, intermediate implant body 805, and distal implant body 806 that interface with the piezoelectric layers 808 and 810 are non-planar. This non-planar configuration ensures uneven distribution of compressive and shear forces across the piezoelectric layers, enhancing the overall stability and promoting enhanced bone growth by virtue of electrical charge generation across the uneven surfaces. The piezoelectric layers 808 and 810 conform to the adjacent non-planar surfaces of the implant bodies, ensuring effective force distribution and electrical charge generation.

The truncated nose 813 at the distal end of the assembly 800 facilitates the insertion process into the SI joint.

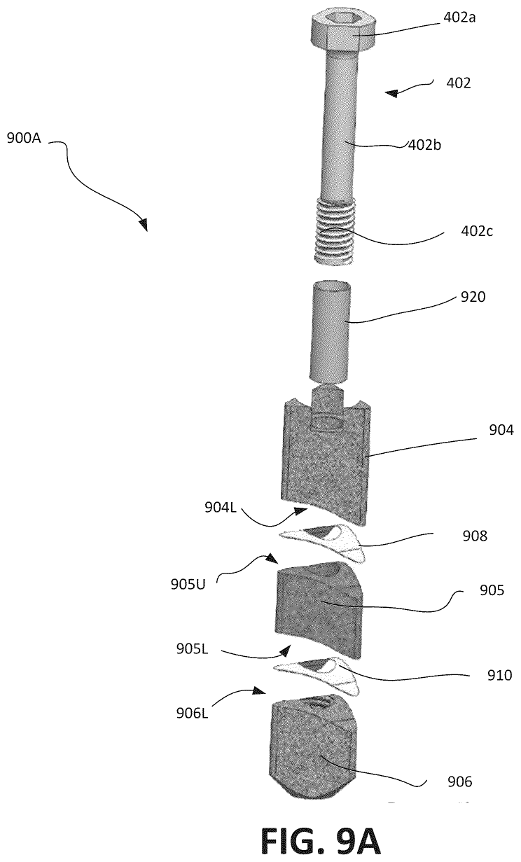

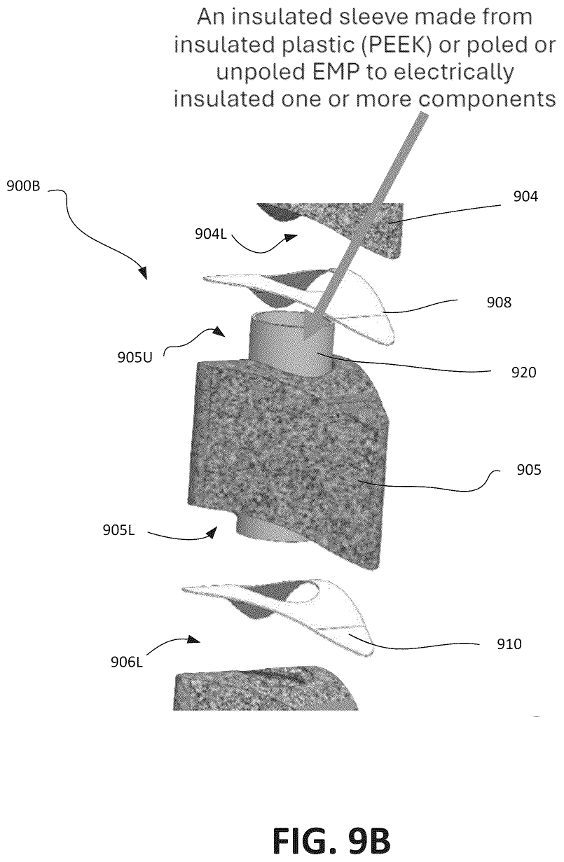

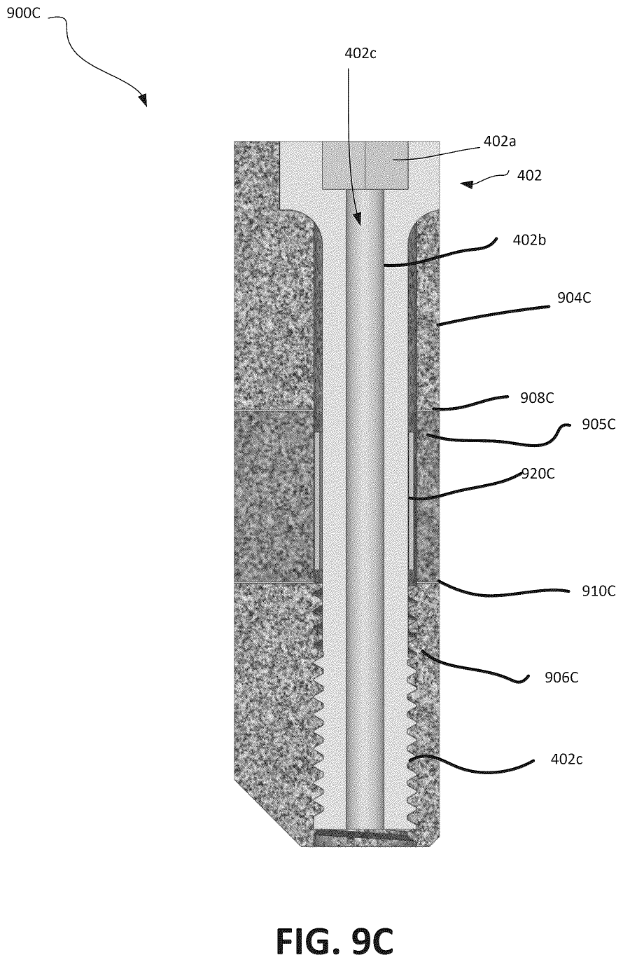

FIG. 9A shows an exploded perspective view of a kit 900A to form an SI joint implant assembly according to a fifth embodiment. The kit 900A further includes an insulating sleeve 920, a proximal implant body 904, an intermediate implant body 905, and a distal implant body 906. Each of these implant bodies includes an electrically conductive material.

The proximal implant body 904 features a lower surface 904L, which is concave. The intermediate implant body 905 has an upper surface 905U that is convex and conforms to the lower surface 904L of the proximal implant body 904. The lower surface 905L of the intermediate implant body 905 is also concave, while the upper surface 906U of the distal implant body 906 is convex and conforms to the lower surface 905L of the intermediate implant body 905.

Between the proximal implant body 904 and the intermediate implant body 905, as well as between the intermediate implant body 905 and the distal implant body 906, are first and second piezoelectric layers 908 and 910, respectively. These piezoelectric layers generate electrical charges when subjected to mechanical stress, which can stimulate bone growth and enhance the stability of the SI joint fusion. The non-planar surfaces of the implant bodies ensure uneven distribution of compressive and shear forces across the piezoelectric layers, enhancing the overall stability and promoting enhanced bone growth. The piezoelectric layers 908 and 910 are designed to conform to the adjacent surfaces of the implant bodies, ensuring effective force distribution and electrical charge generation.

The insulating sleeve 920 is positioned to electrically isolate the lag screw 402 from the implant bodies, maintaining the electrical isolation necessary for the effective functioning of the piezoelectric layers. The assembly of the kit 900A involves aligning the implant bodies and piezoelectric layers to form a continuous structure that can be inserted into the SI joint. The lag screw 402 secures the implant bodies together, maintaining the alignment and structural integrity of the assembly. The sleeve ensures that the intermediate implant body can define an electrical charge polarity that is different from that of the proximal and distal implant bodies.

FIG. 9B shows another exploded perspective view of a kit 900B to form an SI joint implant assembly formed from inserting the sleeve 920 into the lumen of intermediate body 905 prior to assembly of the components to each other. Insulating sleeve 920 may be made from insulated plastic (PEEK) or poled or unpoled EMP to electrically insulate one or more components.

FIG. 9C shows an SI joint implant assembly 900C formed from assembling components of a kit that is a variation of kits 900A or 900B, in that, in FIG. 9C, the surfaces of the implant bodies adjacent the piezoelectric layers are planar rather than convex or concave.

Assembly 900C includes a proximal implant body 904C, an intermediate implant body 905C, and a distal implant body 906C. Each of these implant bodies includes an electrically conductive material.

Between the proximal implant body 904C and the intermediate implant body 905C, as well as between the intermediate implant body 905C and the distal implant body 906C, are first and second piezoelectric layers 908C and 910C, respectively. These piezoelectric layers generate electrical charges when subjected to mechanical stress, which can stimulate bone growth and enhance the stability of the SI joint fusion. The non-planar surfaces of the implant bodies ensure uneven distribution of compressive and shear forces across the piezoelectric layers, enhancing the overall stability and promoting enhanced bone growth. The piezoelectric layers 908C and 910C are designed to conform to the adjacent surfaces of the implant bodies, ensuring effective force distribution and electrical charge generation.

The insulating sleeve 920C is positioned to electrically isolate the lag screw 402 from the implant bodies, maintaining the electrical isolation necessary for the effective functioning of the piezoelectric layers. The assembly of the kit 900CA involves aligning the implant bodies and piezoelectric layers to form a continuous structure that can be inserted into the SI joint. The lag screw 402 secures the implant bodies together, maintaining the alignment and structural integrity of the assembly. The sleeve ensures that the intermediate implant body can define an electrical charge polarity that is different from that of the proximal and distal implant bodies.

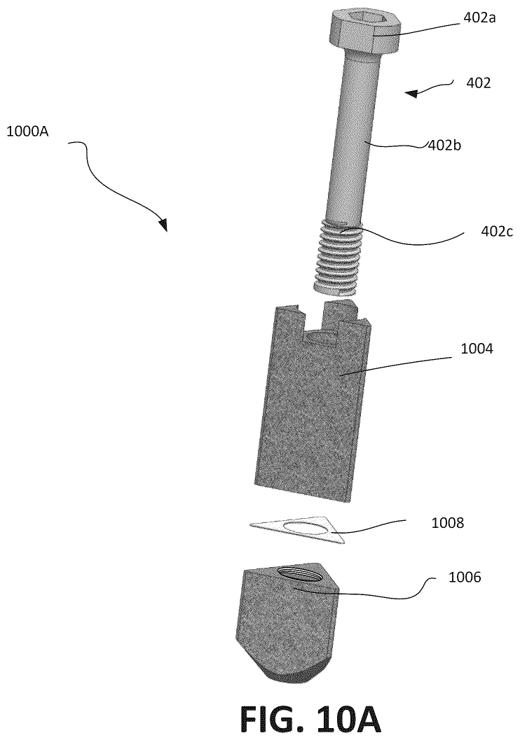

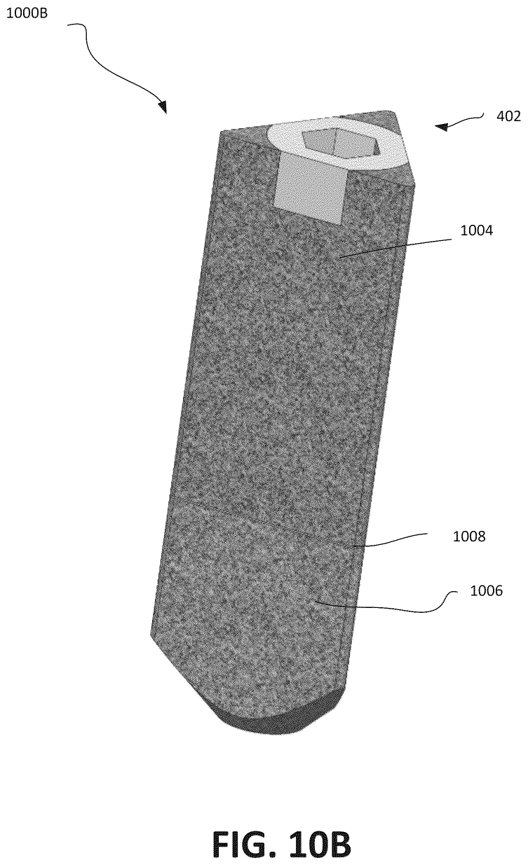

FIG. 10A shows an exploded perspective view of a kit 1000A to form an SI joint implant assembly according to a sixth embodiment.

The kit 1000A includes a proximal implant body 1004, a layer including a piezoelectric material (piezoelectric layer) 1008, and a distal implant body 1006. Each of these implant bodies includes an electrically conductive material. The proximal implant body 1004 and the distal implant body 1006 are designed to interface with the piezoelectric layer 1008, which is positioned between them.

The piezoelectric layer 1008 generates electrical charges when subjected to mechanical stress, which can stimulate bone growth and enhance the stability of the SI joint fusion. The piezoelectric layer 1008 is designed to conform to the adjacent surfaces of the proximal implant body 1004 and the distal implant body 1006, ensuring effective force distribution and electrical charge generation.

The lag screw 402 may include an insulating material to prevent electrical shorting between the proximal implant body 1004 and the distal implant body 1006. This insulation ensures that an electrical charge is generated across the piezoelectric layer 1008, promoting bone growth and enhancing the overall stability of the SI joint fusion.

FIG. 10B shows an SI joint implant assembly 1000B formed from assembling the kit 1000A according to the sixth embodiment.

In this embodiment, the proximal and distal implant bodies 1004 and 1006 are electrically insulated from one another, allowing the single piezoelectric layer 1008 to promote an electric charge across itself through the application of compressive and shear forces to the SI joint implant assembly 1000B.

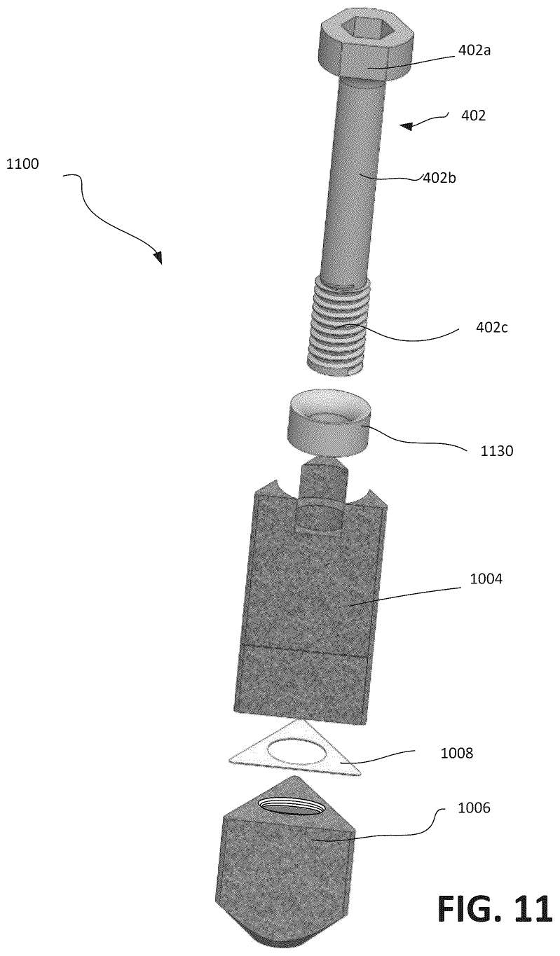

FIG. 11 shows an exploded perspective view of a kit 1100 to form an SI joint implant assembly according to a variation of the embodiments depicted in FIGS. 10A and 10B based on a seventh embodiment. The kit 1100 includes a lag screw 402. The lag screw 402 includes an electrically conductive material.

The kit 1100 further includes a ring 1130, which includes an insulating material. The insulating ring 1130 is positioned to electrically isolate the proximal implant body 1004 from the distal implant body 1006. The proximal implant body 1004 and the distal implant body 1006 are both made from electrically conductive materials. The insulating ring 1130 ensures that the electrical charges generated by the piezoelectric layer 1008 are effectively distributed without interference from the conductive implant bodies.

The through-hole or lumen in the proximal body is oversized to the screw shank such that is does not make mechanical contact with the metallic lag screw which helps maintain electrical insulation between the screw/distal component and the proximal body.

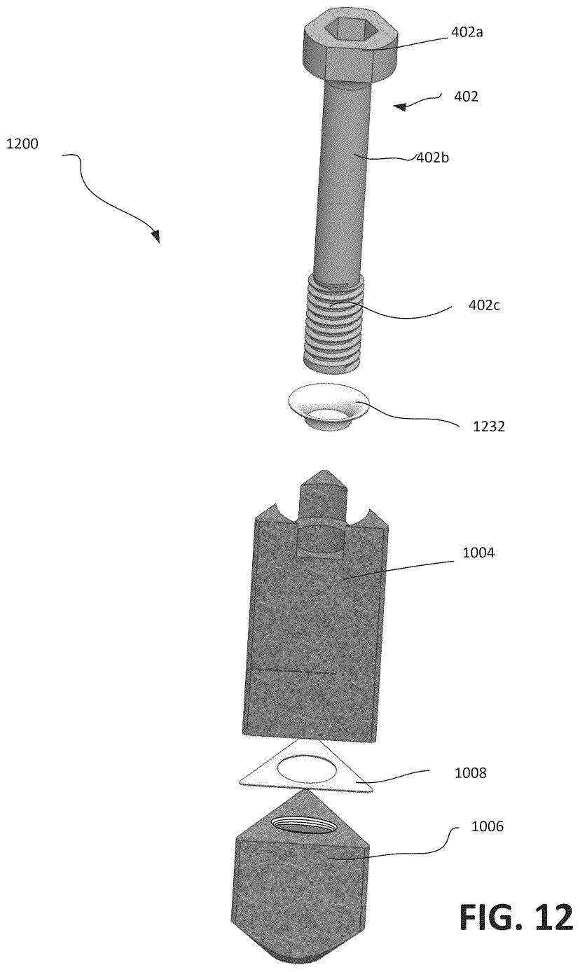

FIG. 12 shows an exploded perspective view of a kit 1200 to form an SI joint implant assembly according to an eighth embodiment.

The kit 1200 includes a screw 402 including an electrically conductive material, and a ring 1232, which includes a piezoelectric material. The piezoelectric ring 1232 provides electrical isolation between the head 402a of the lag screw 402 and the proximal implant body 1004. Additionally, the piezoelectric ring 1232 may create a piezoelectric charge, with one pole distributed to the metallic lag screw 402 and the opposite pole distributed to the proximal implant body 1004.

The proximal implant body 1004 includes an electrically conductive material and is designed to interface with the piezoelectric layer 1008. The piezoelectric layer 1008 is positioned between the proximal implant body 1004 and the distal implant body 1006. The piezoelectric layer 1008 generates electrical charges when subjected to mechanical stress, which can stimulate bone growth and enhance the stability of the SI joint fusion.

The distal implant body 1006 is also made from an electrically conductive material and is designed to interface with the piezoelectric layer 1008. The piezoelectric layer 1008 conforms to the adjacent surfaces of the proximal implant body 1004 and the distal implant body 1006, ensuring effective force distribution and electrical charge generation.

The embodiment of FIG. 12 is comparable to that of FIGS. 4A-4B, 5, 6, 8, 9A-9B, and to embodiments described herein where two piezoelectric layers are provided. In the case of FIG. 12, the screw 402 would act as a distal implant body, the distal implant body 1004 would act as an intermediate implant body, with piezoelectric ring 1232 acting as a first piezoelectric layer, and piezoelectric layer 1008 acting as a second piezoelectric layer, when compared with the embodiments of FIGS. 4A-4B, 5, 6, 8, 9A-9B.

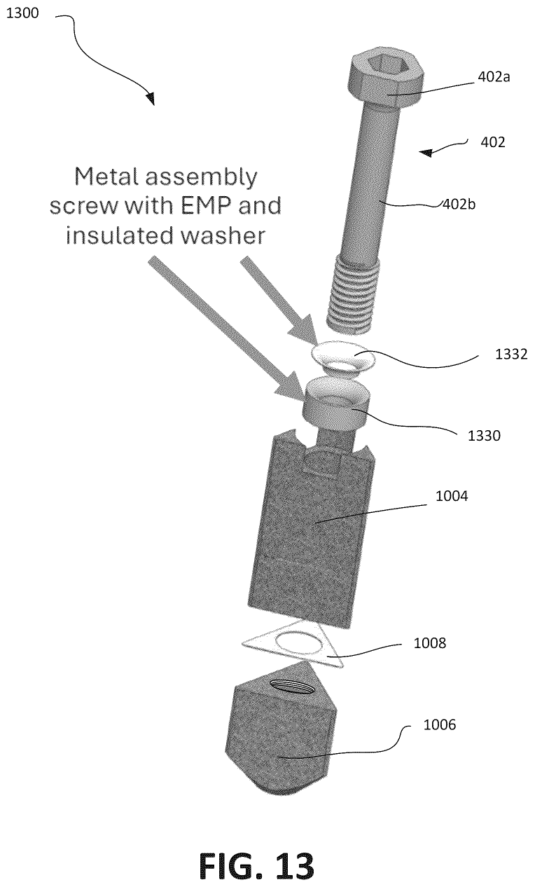

FIG. 13 shows an exploded perspective view of a kit 1300 to form an SI joint implant assembly according to a ninth embodiment. This configuration is similar to the embodiments depicted in FIGS. 11 and 12, except that both an electrically insulating ring 1330 and a piezoelectric ring 1332 are provided together in this embodiment. The lag screw 402 of FIG. 13 includes an electrically conductive material.

The piezoelectric ring 1332 is positioned adjacent to the screw head 402a, and the electrically insulating ring 1330 is positioned adjacent to the proximal implant body 1004 on one side and adjacent to the piezoelectric ring 1332 on the other side. The piezoelectric ring 1332 provides both electrical isolation and the generation of a piezoelectric charge, with one pole distributed to the metallic lag screw 402 and the opposite pole distributed to the proximal implant body 1004. This configuration generates a single pole into the lag screw 402, while the opposite pole is insulated by the insulating ring 1330.

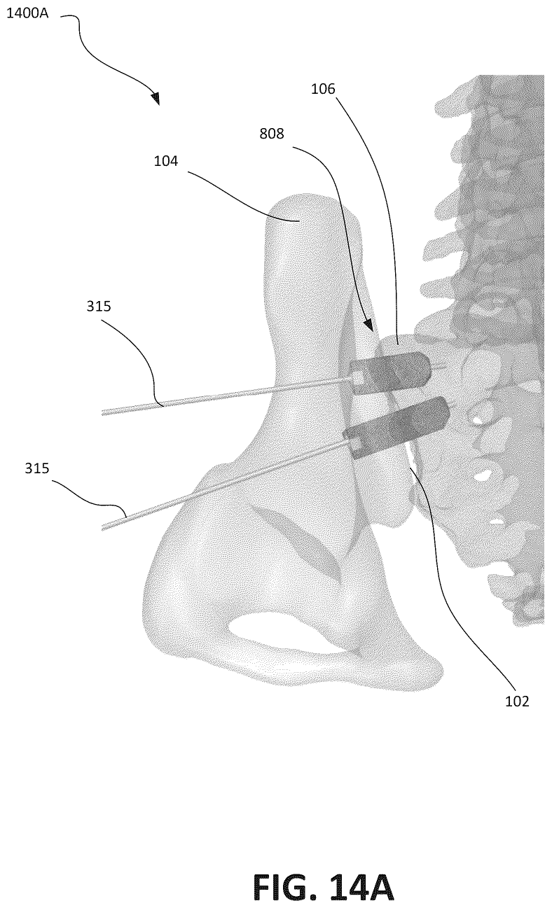

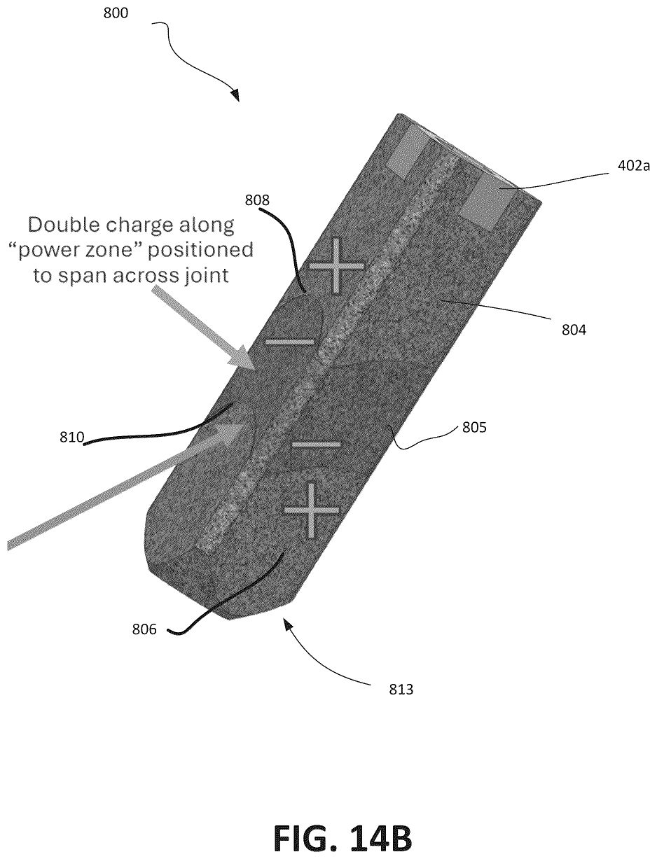

FIG. 14A shows an environment 1400A for an SI joint fusion surgical procedure. The sacroiliac (SI) joint 102 is located between the ilium 104 and the sacrum 106. The SI joint 102 represents a target zone for electrical charge generation. The figure illustrates the placement of SI implant assemblies 800 within the SI joint 102, with the intermediate implant bodies positioned at the joint level or at the target zone.

The SI implant assemblies 80o are designed to stabilize the joint and promote fusion between the ilium 104 and the sacrum 106. The guidance of an SI joint implant assembly to a desired location within the SI joint may be achieved through the use of imaging, for example by SI implant assemblies that include imaging markers thereon (not shown). The guide wires 315 facilitate the precise placement of the implant assemblies 800 during the surgical procedure.

The SI joint implant assembly embodiment 800 depicted in FIG. 14A corresponds to the embodiment shown in FIG. 8, but the SI joint implant assembly could represent any SI joint implant assembly according to embodiments. The use of imaging techniques, such as fluoroscopy, can provide the surgeon with live imaging to ensure proper placement of the implant assemblies 800 within the SI joint 102.

FIG. 14B shows the SI joint implant assembly 800 of FIG. 8, further illustrating the poles created through the piezoelectric effect of forces from the proximal implant body 804, intermediate implant body 805, and distal implant body 806 onto the piezoelectric layers 808 and 810. The piezoelectric effect generates double poles, with a negative pole within the intermediate body 805 and positive poles in each of the proximal implant body 804 and distal implant body 806, although the poles could be reversed in a different embodiment.

The figure emphasizes the “target zone” corresponding to pole boundaries and the space between them, the target zone positioned to span across the joint. This configuration aims to enhance the stimulation of bone growth in the region where the sacrum and ilium meet. The pluses and minuses in the figure represent the electric charge poles, creating a double pole SI joint implant assembly when subjected to mechanical stress.

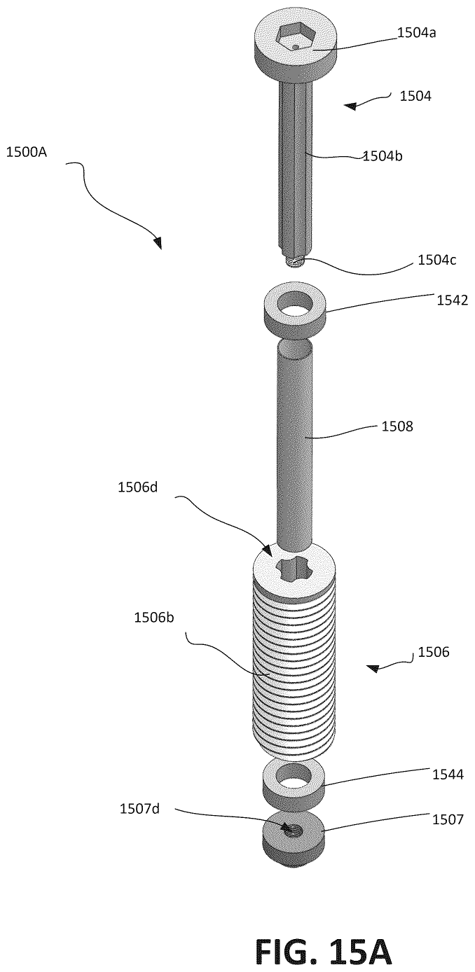

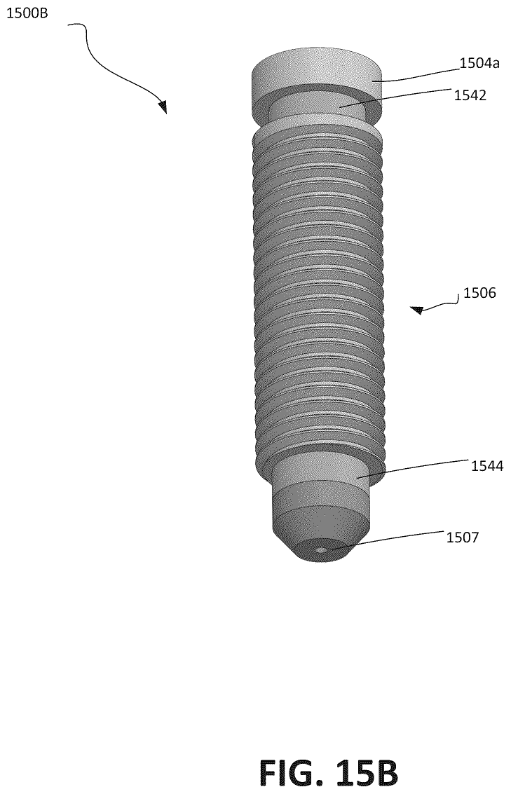

In the embodiments of FIGS. 15A-20B described below, the electrical polarities change in a lateral direction of the final SI joint implant assembly rather than in a longitudinal direction as was the case with previous embodiments as shown in FIGS. 4A-14B. This lateral shift in electrical poles enhances the stimulation of bone growth across the SI joint, even where the application of forces at opposite ends of the SI joint implant assembly after implantation may create opposing moments about a middle (between the two ends) portion of the SI joint implant assembly, promoting enhanced fusion and stability.

FIG. 15A shows an exploded perspective view of a kit 1500A to form an SI joint implant assembly according to a tenth embodiment.

The kit 1500A includes an inner implant body 1504, a sleeve 1508 including a piezoelectric material, an outer implant body 1506, and an implant end body 1507. The inner implant body 1504 comprises an inner implant body head 1504a, an inner implant body shaft portion with an irregular surface 1504b, and an inner implant body threaded portion 1504c. The inner implant body 1504 is designed to be inserted into the outer implant body 1506, which defines a longitudinally extending lumen 1506d to accommodate the sleeve 1508.

The outer implant body 1506 includes a threaded surface 1506b, which provides structural stability and facilitates the secure attachment of the implant assembly within the SI joint. The sleeve 1508, positioned within the lumen 1506d of the outer implant body 1506, includes a piezoelectric material that generates electrical charges when subjected to mechanical stress. This piezoelectric effect stimulates bone growth and enhances the stability of the SI joint fusion.

The kit 1500A further includes a first ring 1542 and a second ring 1544, both of which include an electrically insulating material. The first ring 1542 is positioned between the inner implant body 1504 and the sleeve 1508, while the second ring 1544 is positioned between the outer implant body 1506 and the implant end body 1507. These insulating rings ensure that the electrical charges generated by the piezoelectric sleeve 1508 are effectively distributed without interference from the conductive implant bodies.

The implant end body 1507 includes a threaded lumen 1507d, which engages the inner implant body threaded portion 1504c, securing the entire assembly together. The implant end body 1507 provides additional structural stability and ensures that the implant assembly remains securely in place within the SI joint.

FIG. 15B shows an SI joint implant assembly 1500B formed from assembling the kit 1500A according to the tenth embodiment. The assembly 1500B includes an inner implant body head 1504a, a first ring 1542, an outer implant body 1506, a second ring 1544, and an implant end body 1507.

The first ring 1542, made from an electrically insulating material, is positioned between the inner implant body head 1504a and the outer implant body 1506. This first insulating ring 1542 ensures that the inner implant body head 1504a is electrically isolated from the outer implant body 1506.

The second ring 1544, also made from an electrically insulating material, is positioned between the outer implant body 1506 and the implant end body 1507. This second insulating ring 1544 ensures that the implant end body 1507 is electrically isolated from the outer implant body 1506.

The double insulating rings 1542 and 1544 play a role in electrically isolating the inner implant body from the outer implant body, allowing the formation of an electric charge across the sleeve 1508. This configuration ensures that the electrical charges generated by the piezoelectric sleeve 1508 are effectively distributed without interference from the conductive implant bodies, thereby enhancing the stimulation of bone growth and promoting effective fusion and stability of the SI joint.

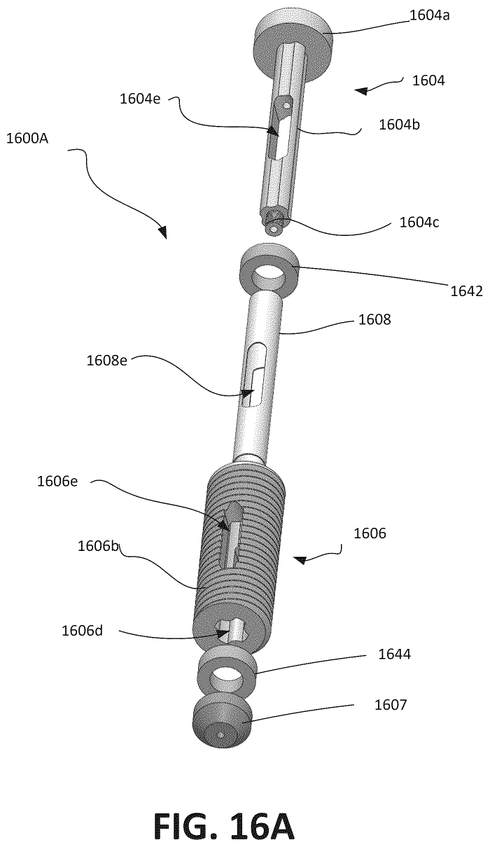

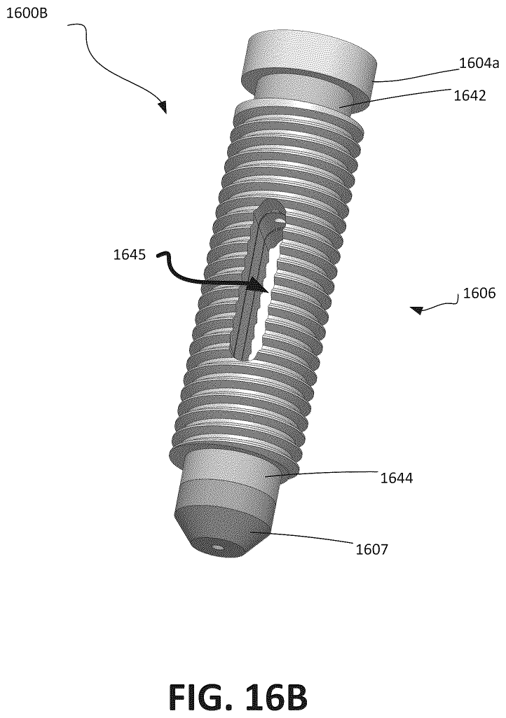

FIG. 16A shows an exploded perspective view of a kit 1600A to form an SI joint implant assembly according to a eleventh embodiment. This embodiment is a variation of the embodiment depicted in FIGS. 15A and 15B, in that the inner implant body, the piezoelectric sleeve and the outer implant body each define a window or fenestration opening therethrough. The kit 1600A includes an inner implant body 1604, a sleeve 1608 including a piezoelectric material, an outer implant body 1606, and an implant end body 1607.

The inner implant body 1604 comprises an inner implant body head 1604a, an inner implant body shaft portion 1604b with an irregular surface, and an inner implant body threaded portion 1604c. The inner implant body 1604 also defines a fenestration opening 1604e extending laterally through the shaft portion 1604b. The inner implant body 1604 is designed to be inserted into the outer implant body 1606, which defines a longitudinally extending lumen 1606d to accommodate the sleeve 1608.

The sleeve 1608, positioned within the lumen 1606d of the outer implant body 1606, includes a piezoelectric material that generates electrical charges when subjected to mechanical stress. The sleeve 1608 also defines a fenestration opening 1608e extending laterally through the structure of the sleeve 1608. This piezoelectric effect stimulates bone growth and enhances the stability of the SI joint fusion.

The outer implant body 1606 includes a threaded surface 1606b, which provides structural stability and facilitates the secure attachment of the implant assembly within the SI joint. The outer implant body 1606 also defines a fenestration opening 1606e extending laterally through the structure of the outer implant body 1606, which aligns/registers with the fenestration openings 1604e and 1608e in the inner implant body 1604 and the sleeve 1608, respectively, in the final assembly, to form fenestration opening 1645 as is shown in FIG. 16B.

The kit 1600A further includes a first ring 1642 and a second ring 1644, both of which include an electrically insulating material. The first ring 1642 is positioned between the inner implant body 1604 and the sleeve 1608, while the second ring 1644 is positioned between the outer implant body 1606 and the implant end body 1607. These insulating rings ensure that the electrical charges generated by the piezoelectric sleeve 1608 are effectively distributed without interference from the conductive implant bodies.

The implant end body 1607 provides additional structural stability and ensures that the implant assembly remains securely in place within the SI joint. The implant end body 1607 is designed to engage the inner implant body threaded portion 1604c, securing the entire assembly together.

FIG. 16B shows an SI joint implant assembly 1600B formed from assembling the kit 1600A according to the eleventh embodiment.

Fenestration opening 1645 extends longitudinally along and laterally through the structure of the SI joint implant assembly. This fenestration opening 1645 is in registration with corresponding fenestration openings in the inner implant body and the piezoelectric sleeve, promoting bone growth laterally across the implant assembly.

The second ring 1644, also made from an electrically insulating material, is positioned between the outer implant body 1606 and the implant end body 1607. This second insulating ring 1644 ensures that the implant end body 1607 is electrically isolated from the outer implant body 1606.

The implant end body 1607 provides additional structural stability and ensures that the implant assembly remains securely in place within the SI joint. The implant end body 1607 is designed to engage the inner implant body, securing the entire assembly together.

The fenestration opening 1645 in the outer implant body 1606 promotes bone growth across the SI joint implant assembly 1600B by providing pathways for bone to grow through the implant. This feature enhances the integration of the implant with the surrounding bone, further stabilizing the joint and promoting effective fusion.

FIG. 16B shows an SI joint implant assembly 1600B formed from assembling the kit 1600A according to the eleventh embodiment. The assembly 1600B includes an inner implant body head 1604a, a first ring 1642, an outer implant body 1606, a second ring 1644, and an implant end body 1607.

The first ring 1642, made from an electrically insulating material, is positioned between the inner implant body head 1604a and the outer implant body 1606. This first insulating ring 1642 ensures that the inner implant body head 1604a is electrically isolated from the outer implant body 1606.

The outer implant body 1606 includes a fenestration opening 1645 extending laterally through its structure. This fenestration opening 1645 aligns with corresponding fenestration openings in the inner implant body and the piezoelectric sleeve, promoting bone growth through the implant assembly. The fenestration opening 1645 provides additional pathways for bone to grow through the implant. This feature enhances the integration of the implant with the surrounding bone, further stabilizing the joint and promoting effective fusion.

The second ring 1644, also made from an electrically insulating material, is positioned between the outer implant body 1606 and the implant end body 1607. This second insulating ring 1644 ensures that the implant end body 1607 is electrically isolated from the outer implant body 1606.

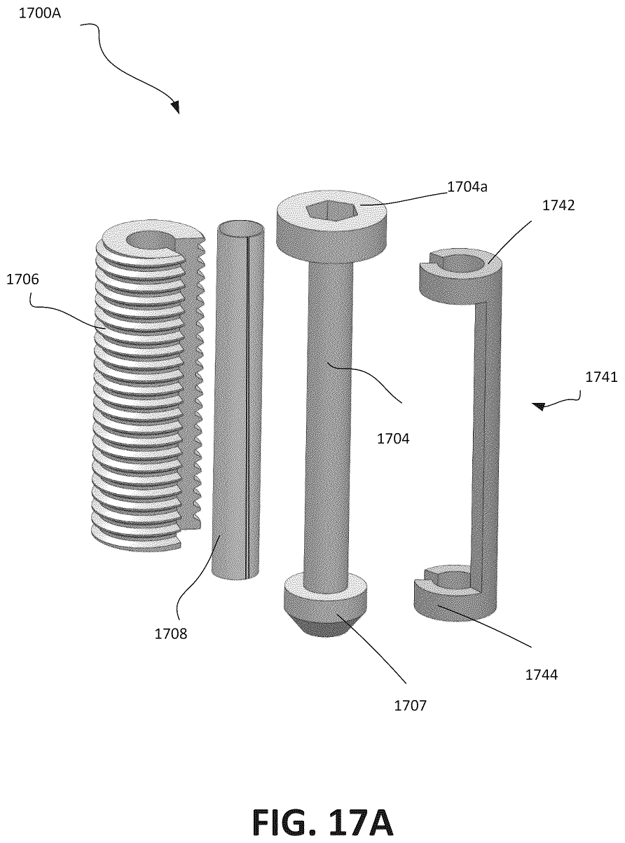

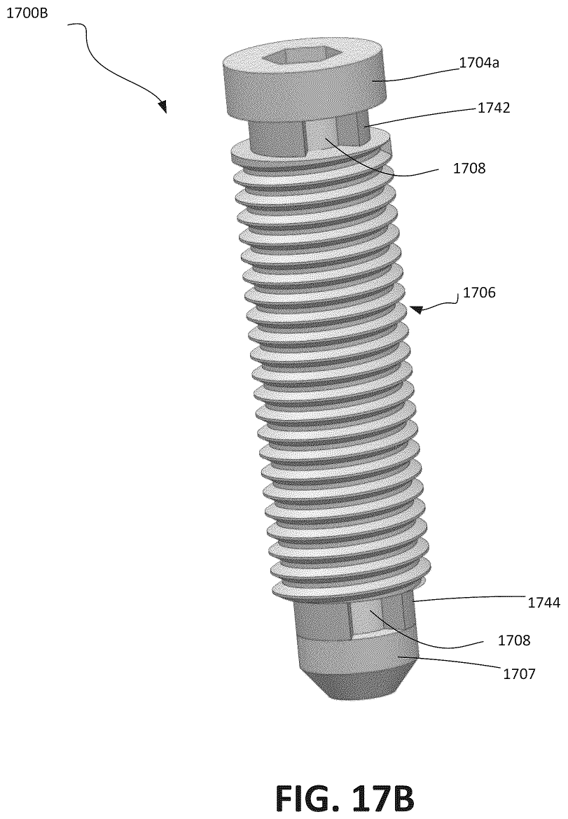

FIG. 17A shows an exploded perspective view of a kit 1700A to form an SI joint implant assembly according to an twelfth embodiment. This embodiment is a variation of the embodiment depicted in FIGS. 16A-16B, in that it presents components of an SI joint implant assembly that are to be assembled together laterally rather than longitudinally as was the case with previously described embodiments of FIGS. 4A-16B.

The kit 1700A includes an inner implant body 1704, an outer implant body 1706, a piezoelectric sleeve 1708, an implant end body 1707, and a bracket 1741 comprising a first ring portion 1742 and a second ring portion 1744.

The inner implant body 1704 includes an inner implant body head 1704a and is designed to be inserted into the outer implant body 1706. The outer implant body 1706 defines a longitudinally extending lumen to accommodate the piezoelectric sleeve 1708. The piezoelectric sleeve 1708 generates electrical charges when subjected to mechanical stress, which can stimulate bone growth and enhance the stability of the SI joint fusion.

The implant end body 1707 provides additional structural stability and ensures that the implant assembly remains securely in place within the SI joint. The bracket 1741, which includes the first ring portion 1742 and the second ring portion 1744, is designed to fit around the shaft of the inner implant body 1704. The insulating rings 1742 and 1744 are part of the bracket 1741 and also include a longitudinal opening therein to fit around the shaft of the inner implant body 1704.

In this embodiment, the electrical poles shift in a lateral direction of the final SI joint implant assembly rather than in a longitudinal direction as with previous embodiments. This lateral shift in electrical poles enhances the stimulation of bone growth across the SI joint, promoting effective fusion and stability.

FIG. 17B shows an SI joint implant assembly 1700B formed from assembling the kit 1700A according to an twelfth embodiment. The assembly 1700B includes an inner implant body head 1704a, a first ring portion 1742, a piezoelectric sleeve 1708, an outer implant body 1706, a second ring portion 1744, and an implant end body 1707.

The first ring portion 1742, made from an electrically insulating material, is positioned between the inner implant body head 1704a and the piezoelectric sleeve 1708. This first insulating ring portion 1742 ensures that the inner implant body head 1704a is electrically isolated from the piezoelectric sleeve 1708.

The second ring portion 1744, also made from an electrically insulating material, is positioned between the piezoelectric sleeve 1708 and the implant end body 1707. This second insulating ring portion 1744 ensures that the implant end body 1707 is electrically isolated from the piezoelectric sleeve 1708.

The double insulating ring portions 1742 and 1744 play a role in electrically isolating the inner implant body head 1704a from the outer implant body 1706, allowing the formation of an electric charge across the piezoelectric sleeve 1708. This configuration ensures that the electrical charges generated by the piezoelectric sleeve 1708 are effectively distributed without interference from the conductive implant bodies, thereby enhancing the stimulation of bone growth and promoting effective fusion and stability of the SI joint.

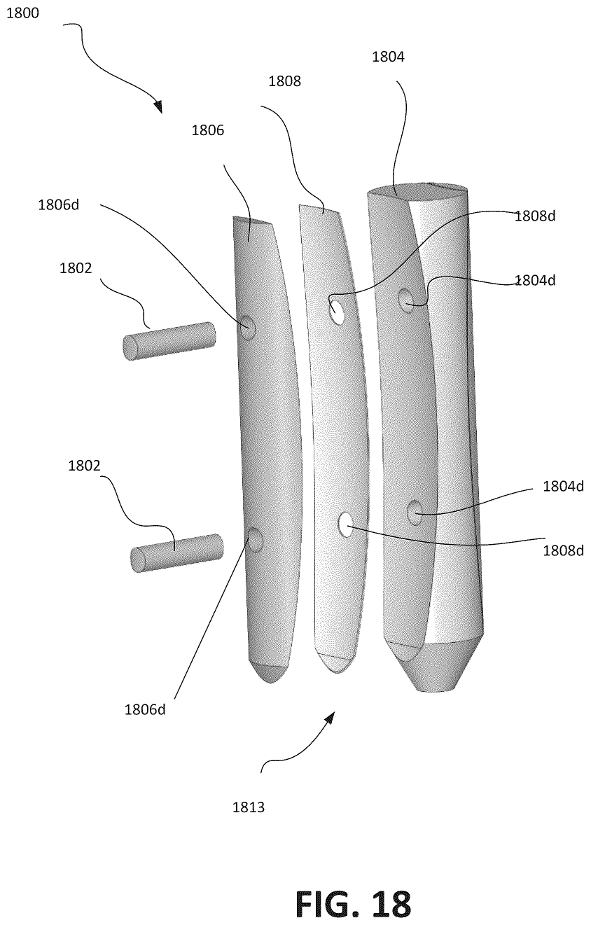

FIG. 18 shows an exploded perspective view of a kit 1800 to form an SI joint implant assembly according to a thirteenth embodiment. The kit 1800 includes a first lateral implant body 1804, a second lateral implant body 1806, a longitudinally extending piezoelectric layer 1808, and pins 1802.

The first lateral implant body 1804 is designed to be positioned on one side of the piezoelectric layer 1808. The first lateral implant body 1804 includes openings 1804d extending laterally through the structure of the first lateral implant body 1804. These openings 1804d are designed to align with corresponding openings in the piezoelectric layer 1808 and the second lateral implant body 1806, facilitating the insertion of the pins 1802.

The second lateral implant body 1806 is designed to be positioned on the opposite side of the piezoelectric layer 1808. The second lateral implant body 1806 includes openings 1806d extending laterally through the structure of the second lateral implant body 1806. These openings 1806d align with the openings 1804d in the first lateral implant body 1804 and the openings 1808d in the piezoelectric layer 1808, allowing the pins 1802 to secure the components together.

The piezoelectric layer 1808 is positioned between the first lateral implant body 1804 and the second lateral implant body 1806. The piezoelectric layer 1808 includes openings 1808d extending laterally through the structure of the piezoelectric layer 1808. These openings 1808d align with the openings 1804d in the first lateral implant body 1804 and the openings 1806d in the second lateral implant body 1806, facilitating the insertion of the pins 1802.

The pins 1802 are designed to extend through the aligned openings 1804d, 1808d, and 1806d in the first lateral implant body 1804, the piezoelectric layer 1808, and the second lateral implant body 1806, respectively. The pins 1802 secure the components together, forming a cohesive SI joint implant assembly.

The first lateral implant body 1804, the second lateral implant body 1806, and the piezoelectric layer 1808 are designed to conform to each other, ensuring effective force distribution and electrical charge generation. The piezoelectric layer 1808 generates electrical charges when subjected to mechanical stress, which can stimulate bone growth and enhance the stability of the SI joint fusion.

The truncated nose 1813 at the distal end of the second lateral implant body 1806 facilitates the insertion process into the SI joint. The non-planar surfaces of the implant bodies ensure uneven distribution of compressive and shear forces across the piezoelectric layer, enhancing the overall stability and promoting bone growth.

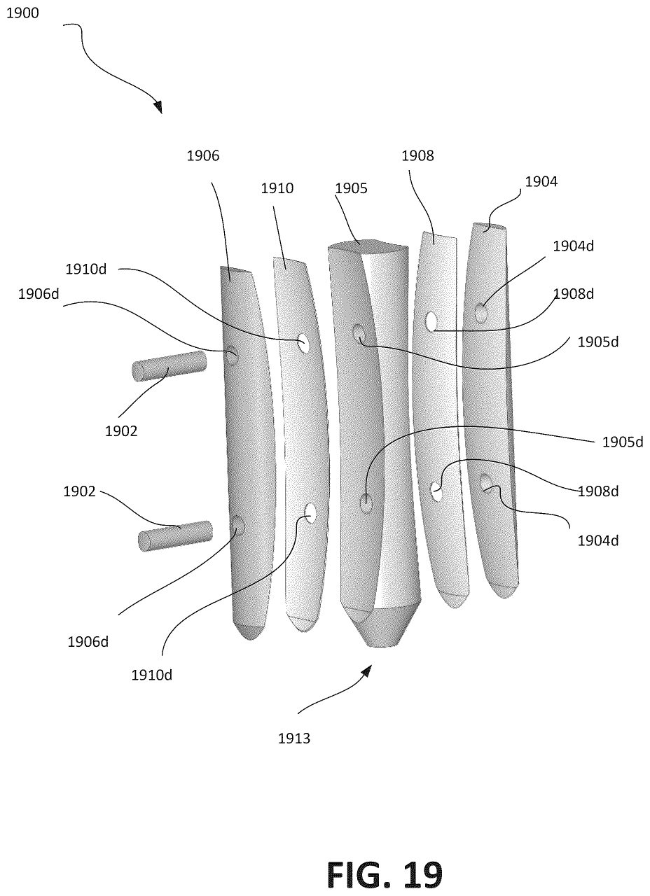

FIG. 19 shows an exploded perspective view of a kit 1900 to form an SI joint implant assembly according to a fourteenth embodiment. The kit 1900 includes a first lateral implant body 1904, a second lateral implant body 1906, an intermediate lateral implant body 1905, a first piezoelectric layer 1908, a second piezoelectric layer 1910, and pins 1902.

The first lateral implant body 1904 is designed to be positioned on one side of the piezoelectric layers 1908 and 1910. The first lateral implant body 1904 includes openings 1904d extending laterally through the structure of the first lateral implant body 1904. These openings 1904d are designed to align with corresponding openings in the piezoelectric layers 1908 and 1910, as well as the intermediate lateral implant body 1905 and the second lateral implant body 1906, facilitating the insertion of the pins 1902.

The second lateral implant body 1906 is designed to be positioned on the opposite side of the piezoelectric layers 1908 and 1910. The second lateral implant body 1906 includes openings 1906d extending laterally through the structure of the second lateral implant body 1906. These openings 1906d align with the openings 1904d in the first lateral implant body 1904, the openings 1908d in the piezoelectric layers 1908 and 1910, and the openings 1905d in the intermediate lateral implant body 1905, allowing the pins 1902 to secure the components together.

The intermediate lateral implant body 1905 is positioned between the first lateral implant body 1904 and the second lateral implant body 1906. The intermediate lateral implant body 1905 includes openings 1905d extending laterally through the structure of the intermediate lateral implant body 1905. These openings 1905d align with the openings 1904d in the first lateral implant body 1904, the openings 1906d in the second lateral implant body 1906, and the openings 1908d in the piezoelectric layers 1908 and 1910, facilitating the insertion of the pins 1902.

The first piezoelectric layer 1908 is positioned between the first lateral implant body 1904 and the intermediate lateral implant body 1905. The first piezoelectric layer 1908 includes openings 1908d extending laterally through the structure of the first piezoelectric layer 1908. These openings 1908d align with the openings 1904d in the first lateral implant body 1904, the openings 1905d in the intermediate lateral implant body 1905, and the openings 1906d in the second lateral implant body 1906, allowing the pins 1902 to secure the components together.

The second piezoelectric layer 1910 is positioned between the intermediate lateral implant body 1905 and the second lateral implant body 1906. The second piezoelectric layer 1910 includes openings 1910d extending laterally through the structure of the second piezoelectric layer 1910. These openings 1910d align with the openings 1904d in the first lateral implant body 1904, the openings 1905d in the intermediate lateral implant body 1905, and the openings 1906d in the second lateral implant body 1906, facilitating the insertion of the pins 1902.

The pins 1902 are designed to extend through the aligned openings 1904d, 1905d, 1906d, 1908d, and 1910d in the first lateral implant body 1904, the intermediate lateral implant body 1905, the second lateral implant body 1906, and the piezoelectric layers 1908 and 1910, respectively. The pins 1902 secure the components together, forming a cohesive SI joint implant assembly.

The first lateral implant body 1904, the second lateral implant body 1906, the intermediate lateral implant body 1905, and the piezoelectric layers 1908 and 1910 are designed to conform to each other, ensuring effective force distribution and electrical charge generation. The piezoelectric layers 1908 and 1910 generate electrical charges when subjected to mechanical stress, which can stimulate bone growth and enhance the stability of the SI joint fusion.

The truncated nose 1913 at the distal end of the intermediate lateral implant body 1905 facilitates the insertion process into the SI joint. The non-planar surfaces of the implant bodies ensure uneven distribution of compressive and shear forces across the piezoelectric layers, enhancing the overall stability and promoting bone growth.

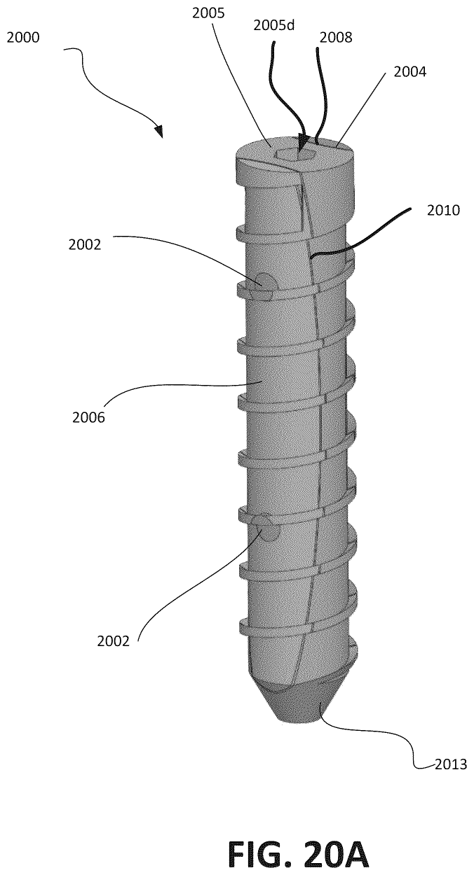

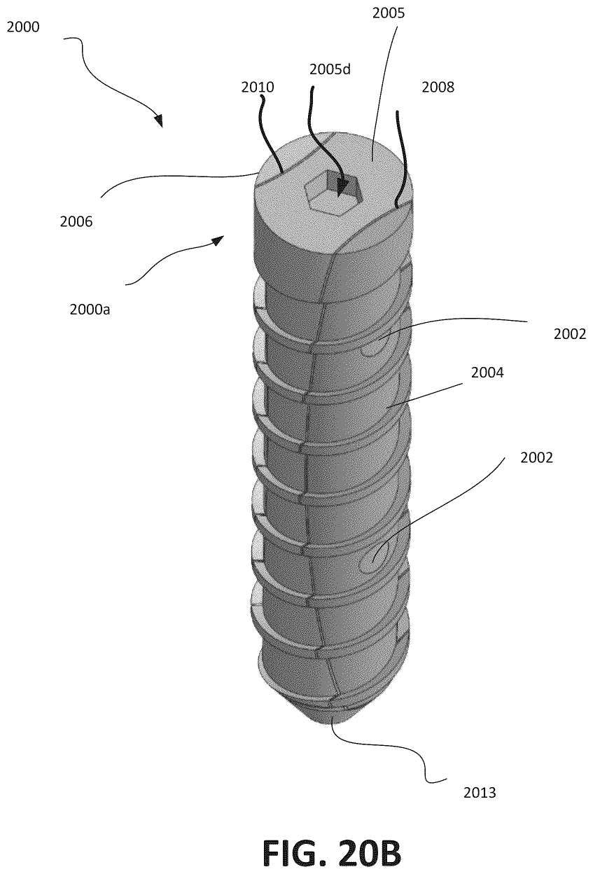

FIGS. 20A and 20B show different perspective views of an SI joint implant assembly 2000 formed from assembling a kit that is a variation of the kit of FIG. 19, in part in that the implant bodies are threaded at outer surfaces thereof to form an SI joint implant assembly that is threaded along an outer surface thereof. SI joint implant assembly thus corresponds to a fifteenth embodiment. The assembly 2000 includes a first lateral implant body 2004, an intermediate lateral implant body 2005, a second lateral implant body 2006, a first piezoelectric layer 2008, a second piezoelectric layer 2010, pins 2002, and a truncated nose 2013.

The first lateral implant body 2004 is positioned at the proximal end of the assembly 2000. The intermediate lateral implant body 2005 is positioned between the first lateral implant body 2004 and the second lateral implant body 2006. The intermediate lateral implant body 2005 includes a lumen 2005d extending longitudinally through the structure of the intermediate lateral implant body 2005, facilitating the alignment and assembly of the implant components.

The first piezoelectric layer 2008 is positioned between the first lateral implant body 2004 and the intermediate lateral implant body 2005. The second piezoelectric layer 2010 is positioned between the intermediate lateral implant body 2005 and the second lateral implant body 2006. These piezoelectric layers generate electrical charges when subjected to mechanical stress, which can stimulate bone growth and enhance the stability of the SI joint fusion.

The pins 2002 extend through the aligned openings in the first lateral implant body 2004, the intermediate lateral implant body 2005, the second lateral implant body 2006, and the piezoelectric layers 2008 and 2010, securing the components together and forming a cohesive SI joint implant assembly. The truncated nose 2013 at the distal end of the second lateral implant body 2006 facilitates the insertion process into the SI joint.

The non-planar surfaces of the implant bodies ensure uneven distribution of compressive and shear forces across the piezoelectric layers, enhancing the overall stability and promoting bone growth. The piezoelectric layers conform to the adjacent surfaces of the implant bodies, ensuring effective force distribution and electrical charge generation.

The threaded outer configuration of the SI joint implant assembly 2000 facilitates the implantation process by providing structural stability and ease of insertion. The tops of the implant bodies together define a head portion, making up the head of the screw-shaped SI joint implant assembly, which aids in the secure placement an

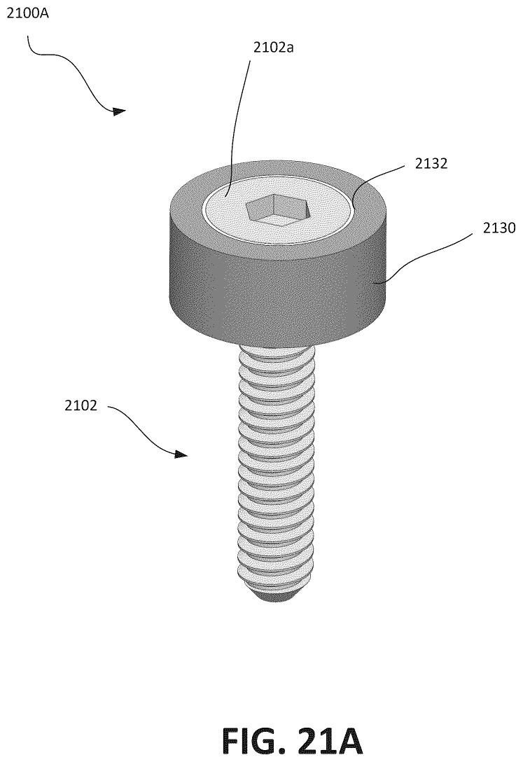

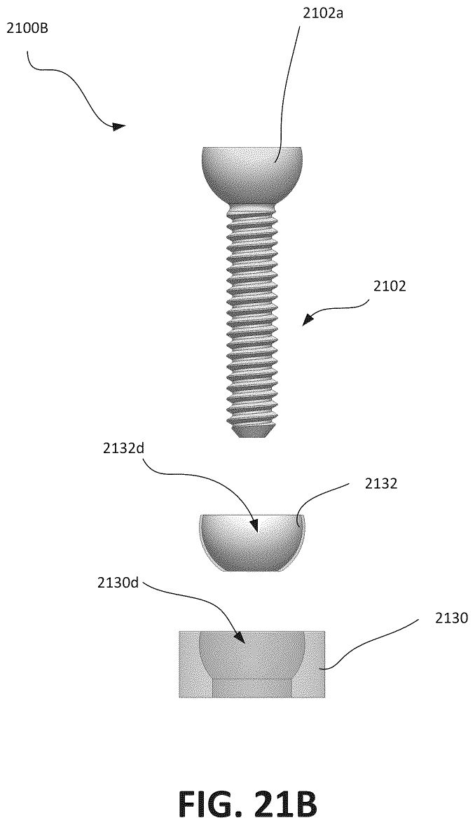

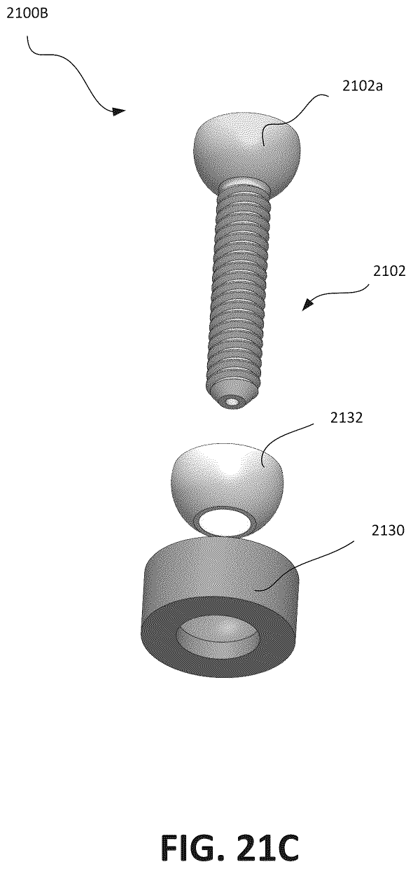

FIG. 21A shows a screw assembly 2100A for use in an orthopedic implant assembly. FIG. 21B shows an exploded perspective view of a kit 2100B to form the screw assembly 2100A for use in an orthopedic implant assembly. FIG. 21C shows an exploded perspective view of a screw assembly 2100B for use in an orthopedic implant assembly.

For example, the screw assembly 2100A may be used as a SI joint implant assembly according to an embodiment The screw assembly 2100A includes a screw 2102, a screw head 2102a, a piezoelectric ring 2132 defining an open cavity 2132d therein, and an electrically insulating ring 2130 defining an open cavity 2130d therein.

The screw 2102 is designed to secure components of an orthopedic implant assembly together. The screw head 2102a is positioned at the proximal end of the screw 2102 and includes a hexagonal recess to accommodate a tool for tightening the screw. The screw head 2102a facilitates the application of torque to the screw 2102 during the assembly process.

The piezoelectric ring 2132 is positioned adjacent to the screw head 2102a. The piezoelectric ring 2132 generates electrical charges when subjected to mechanical stress, which can stimulate bone growth and enhance the stability of the orthopedic implant assembly. The piezoelectric ring 2132 is designed to conform to the shape of the screw head 2102a, ensuring effective force distribution and electrical charge generation.

The electrically insulating ring 2130 is positioned adjacent to the piezoelectric ring 2132. The electrically insulating ring 2130 ensures that the electrical charges generated by the piezoelectric ring 2132 are effectively distributed without interference from the conductive screw 2102. This electrical isolation is necessary for the proper functioning of the piezoelectric ring 2132, as the insulating ring 2130 prevents electrical shorting and ensures that the generated charges stimulate bone growth effectively.

The combination of the screw 2102, the piezoelectric ring 2132, and the electrically insulating ring 2130 in the screw assembly 2100A provides a cohesive structure that enhances the stability and promotes bone growth in the orthopedic implant assembly. The screw assembly 2100A is designed to be used in various orthopedic applications, including sacroiliac joint fusion procedures, where the generation of electrical charges by the piezoelectric ring 2132 can stimulate bone growth and improve the overall efficacy of the implant.

Throughout the specification, and in the claims, the terms “coupled” or “connected” mean a direct or indirect connection, such as a direct electrical, mechanical, or magnetic connection between the elements that are connected or an indirect connection, through one or more passive or active intermediary devices. The term “signal” may refer to at least one current signal, voltage signal, magnetic signal, or data/clock signal. The meaning of “a,” “an,” and “the” include plural references. The meaning of “in” includes “in” and “on.”

The terms “substantially,” “close,” “approximately,” “near,” and “about,” generally refer to being within +/-10% of a target value (unless specifically specified). Unless otherwise specified the use of the ordinal adjectives “first,” “second,” and “third,” etc., to describe a common object, merely indicate that different instances of like objects are being referred to, and are not intended to imply that the objects so described must be in a given sequence, either temporally, spatially, in ranking or in any other manner, and are not intended to imply that the objects so described must necessarily be made of different materials or have different dimensions.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of embodiments has been described with reference to specific example embodiments, various modifications and changes may be made to these embodiments without departing from the broader scope of embodiments of the present disclosure. Such embodiments of the inventive subject matter may be referred to herein, individually or collectively, by the term “invention” merely for convenience and without intending to voluntarily limit the scope of this application to any single disclosure or inventive concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

It will also be understood that, although the terms “first,” “second,” and so forth may be used herein to describe various elements, these elements should not be limited by these terms. These terms are only used to distinguish one element from another. For example, a first contact could be termed a second contact, and, similarly, a second contact could be termed a first contact, without departing from the scope of the present example embodiments. The first contact and the second contact are both contacts, but they are not the same contact.

As used herein the terms “top,” “bottom,” “upper,” “lower,” “lowermost,” and “uppermost” when used in relationship to one or more elements are intended to convey a relative rather than absolute physical configuration. Thus, an element described as an “uppermost element” or a “top element” in a device may instead form the “lowermost element” or “bottom element” in the device when the device is inverted. Similarly, an element described as the “lowermost element” or “bottom element” in the device may instead form the “uppermost element” or “top element” in the device when the device is inverted.

The description may use perspective-based descriptions such as top/bottom, in/out, over/under, and the like. Such descriptions are merely used to facilitate the discussion and are not intended to restrict the application of embodiments described herein to any particular orientation.

As used in the description of the example embodiments and the appended examples, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

### EXAMPLES

Illustrative examples of the technologies described throughout this disclosure are provided below. Embodiments of these technologies may include any one or more, and any combination of, the examples described below. In some embodiments, at least one of the systems or components set forth in one or more of the preceding figures may be configured as set forth in the following examples.

Example 1 includes a sacroiliac (SI) joint implant assembly, comprising: a first implant body; a second implant body attached to the first implant body, wherein the first implant body and the second implant body each comprise an electrically conductive material; a piezoelectric layer between the first implant body and the second implant body and to generate electrical charges when subjected to mechanical stress through at least one of the first implant body or the second implant body.

Example 2 includes the subject matter of Example 1, further including a fastener to fasten the first implant body and the second implant body together.

Example 3 includes the subject matter of Example 1, wherein the fastener is a lag screw.

Example 4 includes the subject matter of Example 2, wherein the fastener includes a pin.

Example 5 includes the subject matter of any one of Examples 2-3, wherein the fastener is cannulated to accommodate a guide wire therein.

Example 6 includes the subject matter of any one of Examples 2-4, wherein the fastener includes an electrically conductive material.

Example 7 includes the subject matter of any one of Examples 2-5, wherein the fastener includes an electrically insulating material.

Example 8 includes the subject matter of any one of Examples 1-7, wherein the first implant body includes a first non-planar inner surface facing the piezoelectric layer, and the second implant body includes a second non-planar inner surface facing the piezoelectric layer.

Example 9 includes the subject matter of any one of Examples 8, wherein the first inner surface defines first undulations facing the piezoelectric layer, and the second implant body includes a second inner surface defining second undulations facing the piezoelectric layer.

Example 10 includes the subject matter of any one of Examples 8-9, wherein the first non-planar inner surface and the second non-planar inner surface conform to one another.

Example 11 includes the subject matter of Example 10, wherein the piezoelectric layer has a first surface that conforms to the first non-planar inner surface, and a second surface that conforms to the second non-planar inner surface.

Example 12 includes the subject matter of any one of Examples 2-10, further comprising an insulating sleeve electrically isolating the fastener from the first implant body and from the second implant body.

Example 13 includes the subject matter of Example 12, wherein the insulating sleeve includes polyether ether ketone (PEEK).

Example 14 includes the subject matter of any one of Examples 1-13, wherein the piezoelectric layer includes a piezoelectric polymer.

Example 15 includes the subject matter of any one of Examples 1-14, wherein the piezoelectric layer includes polyvinylidene fluoride (PVDF).

Example 16 includes the subject matter of any one of Examples 1-15, wherein the first implant body and the second implant body are porous to allow bone ingrowth.

Example 17 includes the subject matter of any one of Examples 1-16, wherein the first implant body and the second implant body define fenestration openings to allow bone growth therein.

Example 18 includes the subject matter of any one of Examples 1-17, wherein the first implant body and the second implant body include titanium.

Example 19 includes the subject matter of any one of Examples 1-18, wherein the first implant body and the second implant body are 3D printed.

Example 20 includes the subject matter of any one of Examples 1-18, wherein the first implant body and the second implant body are machined.

Example 21 includes the subject matter of any one of Examples 1-20, wherein the first implant body and the second implant body include a biocompatible material.

Example 22 includes the subject matter of any one of Examples 2-21, wherein the fastener includes a biocompatible material.

Example 23 includes the subject matter of any one of Examples 1-22, wherein the first implant body and the second implant body include imaging markers to facilitate precise placement during a surgical procedure.

Example 24 includes the subject matter of any one of Examples 1-23, wherein the SI joint assembly is screw-shaped to facilitate insertion into an SI joint.

Example 25 includes the subject matter of any one of Examples 2-24, wherein the piezoelectric layer includes a first piezoelectric layer and a second piezoelectric layer, the SI joint assembly further including an intermediate implant body between the first piezoelectric layer and the second piezoelectric layer.

Example 26 includes the subject matter of Example 25, further including an insulating sleeve between the fastener and the intermediate implant body.

Example 27 includes the subject matter of Example 25, wherein the piezoelectric layer further includes a third piezoelectric layer and a fourth piezoelectric layer, the SI joint assembly further including a first intermediate implant body between the first and second piezoelectric layers, a second intermediate implant body between the third and fourth piezoelectric layers, and a middle implant body between the second and third piezoelectric layers.

Example 28 includes the subject matter of any one of Examples 2-27, further including an insulating ring between the fastener and the first implant body.

Example 29 includes the subject matter of any one of Examples 2-28, further including a piezoelectric ring between the fastener and the first implant body.

Example 30 includes the subject matter of Example 28, further including a piezoelectric ring between the fastener and the insulating ring.

Example 31 includes the subject matter of any one of Examples 1-30, wherein the SI joint assembly has a substantially triangular configuration.

Example 32 includes a kit to form a sacroiliac (SI) joint implant assembly, the kit comprising: a first implant body; a second implant body to be attached to the first implant body, wherein the first implant body and the second implant body each comprise an electrically conductive material; and a piezoelectric layer sized to be between the first implant body and the second implant body in the SI joint assembly, the piezoelectric layer to generate electrical charges when subjected to mechanical stress.

Example 33 includes the subject matter of Example 32, further including a fastener to fasten the first implant body and the second implant body together to form the SI joint implant assembly.

Example 34 includes the subject matter of Example 33, wherein the fastener includes a lag screw.

Example 35 includes the subject matter of Example 33, wherein the fastener includes a pin.

Example 36 includes the subject matter of any one of Examples 33-35, wherein the fastener is cannulated to accommodate a guide wire therein.

Example 37 includes the subject matter of any one of Examples 33-36, further comprising an insulating sleeve to electrically isolate the fastener from the first implant body and from the second implant body.

Example 38 includes a method to form a sacroiliac (SI) joint implant assembly, comprising: providing a first implant body; providing a second implant body; positioning a piezoelectric layer between the first implant body and the second implant body; and securing the first implant body and the second implant body together with a fastener to hold the piezoelectric layer therebetween.

Example 39 includes the subject matter of Example 38, wherein providing the first implant body and providing the second implant body includes using three-dimensional (3D) printing.

Example 40 includes the subject matter of Example 38, wherein providing the fastener includes using machining.

What is claimed is:

1. A sacroiliac (SI) joint implant assembly, comprising:
a first implant body;
a second implant body attached to the first implant body, wherein the first implant body and the second implant body each comprise an electrically conductive material;
a piezoelectric layer between the first implant body and the second implant body and to generate electrical charges when subjected to mechanical stress through at least one of the first implant body or the second implant body.

2. The SI joint implant assembly of claim 1, further including a fastener to fasten the first implant body and the second implant body together.

3. The SI joint implant assembly of claim 2, wherein the fastener is a lag screw.

4. The SI joint implant assembly of claim 2, wherein the fastener includes a pin.

5. The SI joint implant assembly of claim 2, wherein the fastener is cannulated to accommodate a guide wire therein.

6. The SI joint implant assembly of claim 2, wherein the fastener includes an electrically conductive material.

7. The SI joint implant assembly of claim 2, wherein the fastener includes an electrically insulating material.

8. The SI joint implant assembly of claim 1, wherein the first implant body includes a first non-planar inner surface facing the piezoelectric layer, and the second implant body includes a second non-planar inner surface facing the piezoelectric layer.

9. The SI joint implant assembly of claim 8, wherein the first inner surface defines first undulations facing the piezoelectric layer, and the second implant body includes a second inner surface defining second undulations facing the piezoelectric layer.

10. The SI joint implant assembly of claim 8, wherein the first non-planar inner surface and the second non-planar inner surface conform to one another.

11. The SI joint implant assembly of claim 10, wherein the piezoelectric layer has a first surface that conforms to the first non-planar inner surface, and a second surface that conforms to the second non-planar inner surface.

12. The SI joint implant assembly of claim 2, further comprising an insulating sleeve electrically isolating the fastener from the first implant body and from the second implant body.

13. The SI joint implant assembly of claim 12, wherein the insulating sleeve includes polyether ether ketone (PEEK).

14. The SI joint implant assembly of claim 1, wherein the piezoelectric layer includes a piezoelectric polymer.

15. The SI joint implant assembly of claim 1, wherein the piezoelectric layer includes polyvinylidene fluoride (PVDF).

16. The SI joint implant assembly of claim 1, wherein the first implant body and the second implant body are porous to allow bone ingrowth.

17. The SI joint implant assembly of claim 1, wherein the first implant body and the second implant body define fenestration openings to allow bone growth therein.

18. The SI joint implant assembly of claim 1, wherein the first implant body and the second implant body include titanium.

19. The SI joint implant assembly of claim 1, wherein the first implant body and the second implant body are 3D printed.

20. The SI joint implant assembly of claim 1, wherein the first implant body and the second implant body are machined.

21. The SI joint implant assembly of claim 1, wherein the first implant body and the second implant body include a biocompatible material.

22. The SI joint implant assembly of claim 2, wherein the fastener includes a biocompatible material.

23. The SI joint implant assembly of claim 1, wherein the first implant body and the second implant body include imaging markers to facilitate precise placement during a surgical procedure.

24. The SI joint implant assembly of claim 1, wherein the SI joint assembly is screw-shaped to facilitate insertion into an SI joint.

25. The SI joint implant assembly of claim 2, wherein the piezoelectric layer includes a first piezoelectric layer and a second piezoelectric layer, the SI joint assembly further including an intermediate implant body between the first piezoelectric layer and the second piezoelectric layer.

26. The SI joint implant assembly of claim 25, further including an insulating sleeve between the fastener and the intermediate implant body.

27. The SI joint implant assembly of claim 25, wherein the piezoelectric layer further includes a third piezoelectric layer and a fourth piezoelectric layer, the SI joint assembly further including a first intermediate implant body between the first and second piezoelectric layers, a second intermediate implant body between the third and fourth piezoelectric layers, and a middle implant body between the second and third piezoelectric layers.

28. The SI joint implant assembly of claim 2, further including an insulating ring between the fastener and the first implant body.

29. The SI joint implant assembly of claim 2, further including a piezoelectric ring between the fastener and the first implant body.

30. The SI joint implant assembly of claim 28, further including a piezoelectric ring between the fastener and the insulating ring.

31. The SI joint implant assembly of claim 1, wherein the SI joint assembly has a substantially triangular configuration.

32. A kit to form a sacroiliac (SI) joint implant assembly, the kit comprising:
a first implant body;
a second implant body to be attached to the first implant body, wherein the first implant body and the second implant body each comprise an electrically conductive material; and
a piezoelectric layer sized to be between the first implant body and the second implant body in the SI joint assembly, the piezoelectric layer to generate electrical charges when subjected to mechanical stress.

33. The kit of claim 32, further including a fastener to fasten the first implant body and the second implant body together to form the SI joint implant assembly.

34. The kit of claim 33, wherein the fastener includes a lag screw.

35. The kit of claim 33, wherein the fastener includes a pin.

36. The kit of claim 33, wherein the fastener is cannulated to accommodate a guide wire therein.

37. The kit of claim 33, further comprising an insulating sleeve to electrically isolate the fastener from the first implant body and from the second implant body.

38. A method to form a sacroiliac (SI) joint implant assembly, comprising:
providing a first implant body;
providing a second implant body;
positioning a piezoelectric layer between the first implant body and the second implant body; and
securing the first implant body and the second implant body together with a fastener to hold the piezoelectric layer therebetween.

39. The method of claim 38, wherein providing the first implant body and providing the second implant body includes using three-dimensional (3D) printing.

40. The method of claim 38, wherein providing the fastener includes using machining.
